SEQUENCE LISTING (30 sequences)

SEQ ID NO: 1 - protein, 4 aa, Artificial Sequence
  Synthetic
LPAC

SEQ ID NO: 2 - protein, 4 aa, Artificial Sequence
  Synthetic
QAGR

SEQ ID NO: 3 - protein, 2 aa, Artificial Sequence
  Synthetic
  REPEAT (1)..(2): May repeat 5, 10, 15, 20, 25, 30, 35, 40, or more than 40 times
CP

SEQ ID NO: 4 - protein, 2 aa, Artificial Sequence
  Synthetic
  REPEAT (1)..(2): May repeat 5, 10, 15, 20, 25, 30, 35, 40, or more than 40 times
GA

SEQ ID NO: 5 - protein, 2 aa, Artificial Sequence
  Synthetic
  REPEAT (1)..(2): May repeat 5, 10, 15, 20, 25, 30, 35, 40, or more than 40 times
GP

SEQ ID NO: 6 - protein, 2 aa, Artificial Sequence
  Synthetic
  REPEAT (1)..(2): May repeat 5, 10, 15, 20, 25, 30, 35, 40, or more than 40 times
PR

SEQ ID NO: 7 - protein, 2 aa, Artificial Sequence
  Synthetic
  REPEAT (1)..(2): May repeat 5, 10, 15, 20, 25, 30, 35, 40, or more than 40 times
GR

SEQ ID NO: 8 - protein, 2 aa, Artificial Sequence
  Synthetic
  REPEAT (1)..(2): May repeat 5, 10, 15, 20, 25, 30, 35, 40, or more than 40 times
PA

SEQ ID NO: 9 - protein, 1 aa, Artificial Sequence
  Synthetic
  REPEAT (1)..(1): May repeat 5, 10, 15, 20, 25, 30, 35, 40, or more than 40 times
A

SEQ ID NO: 10 - protein, 1 aa, Artificial Sequence
  Synthetic
  REPEAT (1)..(1): May repeat 5, 10, 15, 20, 25, 30, 35, 40, or more than 40 times
G

SEQ ID NO: 11 - protein, 1 aa, Artificial Sequence
  Synthetic
  REPEAT (1)..(1): May repeat 5, 10, 15, 20, 25, 30, 35, 40, or more than 40 times
S

SEQ ID NO: 12 - protein, 1 aa, Artificial Sequence
  Synthetic
  REPEAT (1)..(1): May repeat 5, 10, 15, 20, 25, 30, 35, 40, or more than 40 times
C

SEQ ID NO: 13 - protein, 1 aa, Artificial Sequence
  Synthetic
  REPEAT (1)..(1): May repeat 5, 10, 15, 20, 25, 30, 35, 40, or more than 40 times
Q

SEQ ID NO: 14 - protein, 2 aa, Artificial Sequence
  Synthetic
  REPEAT (1)..(2): May repeat 5, 10, 15, 20, 25, 30, 35, 40, or more than 40 times
GD

SEQ ID NO: 15 - protein, 2 aa, Artificial Sequence
  Synthetic
  REPEAT (1)..(2): May repeat 5, 10, 15, 20, 25, 30, 35, 40, or more than 40 times
GE

SEQ ID NO: 16 - protein, 2 aa, Artificial Sequence
  Synthetic
  REPEAT (1)..(2): May repeat 5, 10, 15, 20, 25, 30, 35, 40, or more than 40 times
GQ

SEQ ID NO: 17 - protein, 2 aa, Artificial Sequence
  Synthetic
  REPEAT (1)..(2): May repeat 5, 10, 15, 20, 25, 30, 35, 40, or more than 40 times
GT

SEQ ID NO: 18 - protein, 1 aa, Artificial Sequence
  Synthetic
  REPEAT (1)..(1): May repeat 5, 10, 15, 20, 25, 30, 35, 40, or more than 40 times
L

SEQ ID NO: 19 - protein, 2 aa, Artificial Sequence
  Synthetic
  REPEAT (1)..(2): May repeat 5, 10, 15, 20, 25, 30, 35, 40, or more than 40 times
LP

SEQ ID NO: 20 - protein, 4 aa, Artificial Sequence
  Synthetic
  REPEAT (1)..(4): May repeat 5, 10, 15, 20, 25, 30, 35, 40, or more than 40 times
LPAC

SEQ ID NO: 21 - protein, 2 aa, Artificial Sequence
  Synthetic
  REPEAT (1)..(2): May repeat 5, 10, 15, 20, 25, 30, 35, 40, or more than 40 times
LS

SEQ ID NO: 22 - protein, 1 aa, Artificial Sequence
  Synthetic
  REPEAT (1)..(1): May repeat 5, 10, 15, 20, 25, 30, 35, 40, or more than 40 times
P

SEQ ID NO: 23 - protein, 4 aa, Artificial Sequence
  Synthetic
  REPEAT (1)..(4): May repeat 5, 10, 15, 20, 25, 30, 35, 40, or more than 40 times
QAGR

SEQ ID NO: 24 - protein, 2 aa, Artificial Sequence
  Synthetic
  REPEAT (1)..(2): May repeat 5, 10, 15, 20, 25, 30, 35, 40, or more than 40 times
RE

SEQ ID NO: 25 - protein, 2 aa, Artificial Sequence
  Synthetic
  REPEAT (1)..(2): May repeat 5, 10, 15, 20, 25, 30, 35, 40, or more than 40 times
SP

SEQ ID NO: 26 - protein, 2 aa, Artificial Sequence
  Synthetic
  REPEAT (1)..(2): May repeat 5, 10, 15, 20, 25, 30, 35, 40, or more than 40 times
VP

SEQ ID NO: 27 - protein, 2 aa, Artificial Sequence
  Synthetic
  REPEAT (1)..(2): May repeat 5, 10, 15, 20, 25, 30, 35, 40, or more than 40 times
FP

SEQ ID NO: 28 - protein, 2 aa, Artificial Sequence
  Synthetic
  REPEAT (1)..(2): May repeat 5, 10, 15, 20, 25, 30, 35, 40, or more than 40 times
GK

SEQ ID NO: 29 - protein, 4 aa, Artificial Sequence
  Synthetic
LLLL

SEQ ID NO: 30 - protein, 5 aa, Artificial Sequence
  Synthetic
WNGME

(19) United States
(12) Patent Application Publication
(10) Pub. No.: US 2023/0218730 A1
(43) Pub. Date: Jul. 13, 2023

(54) VACCINE THERAPY FOR RAN PROTEIN DISEASES

(71) Applicant: University of Florida Research Foundation, Incorporated, Gainesville, FL (US)

(72) Inventors: Laura Ranum, Gainesville, FL (US); Lauren A. Laboissonniere, Gainesville, FL (US)

(73) Assignee: University of Florida Research Foundation, Incorporated, Gainesville, FL (US)

(21) Appl. No.: 17/762,543

(22) PCT Filed: Sep. 18, 2020
(86) PCT No.: PCT/US2020/051670

§ 371 Date: Mar. 22, 2022

Related U.S. Application Data
(60) Provisional application No. 62/904,612, filed on Sep. 23, 2019.

(51) Int. Cl. A61K 39/00; C07K 14/47; A61P 25/28; A61K 39/39

(52) U.S. Cl. CPC A61K 39/0007; C07K 14/4702; A61P 25/28; A61K 39/39; A61K 2039/545; A61K 2039/53; A61K 2039/55561; A61K 2039/55516

(57) ABSTRACT

Aspects of the disclosure relate to compositions and methods for eliciting (or enhancing) anti-repeat-associated non-ATG (RAN) protein antibody expression or production in a subject. Administration of the compositions according to the methods of the present disclosure may in some embodiments result in decreased levels of RAN protein expression and/or aggregation. Such compositions and methods may therefore be useful for the treatment of diseases and disorders known to be associated with RAN proteins.

### RELATED APPLICATIONS

This application is a national stage filing under 35 U.S.C. § 371 of International Patent Application Serial No. PCT/US2020/051670, filed Sep. 18, 2020, which claims the benefit under 35 U.S.C. § 119(e) of the filing date of U.S. provisional Application Ser. No. 62/904,612, filed Sep. 23, 2019, entitled “VACCINE THERAPY FOR RAN PROTEIN DISEASES”, the entire contents of each of which are incorporated herein by reference in their entirety.

### FEDERALLY SPONSORED RESEARCH

This invention was made with government support under Grant No. R01 NS098819 awarded by The National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Feb. 17, 2022, is named U120270071US01-SEQ-KSB.txt and is 7,214 bytes in size.

### BACKGROUND

Microsatellite repeat expansions are known to cause more than forty neurodegenerative diseases and disorders. In a growing number of these diseases and disorders, expansion mutations have been shown to undergo a novel type of protein translation that occurs in multiple reading frames and does not require a canonical AUG initiation codon. This type of translation is called repeat associated non-ATG (RAN) translation and the proteins that are produced are called RAN proteins. There is growing evidence that RAN proteins are toxic and contribute to a growing number of diseases and disorders, including, but not limited to, amyotrophic lateral sclerosis (ALS), frontotemporal dementia (FTD), myotonic dystrophy type 1 (DM1), myotonic dystrophy type 2 (DM2), Huntington's disease (HD), Alzheimer's disease (AD), and Fragile X Tremor Ataxia Syndrome (FXTAS).

### SUMMARY

Aspects of the disclosure relate to compositions and methods for eliciting (or enhancing) anti-RAN protein antibody expression or production in a subject. The disclosure is based, in part, on compositions for vaccinating a subject to produce antibodies against certain RAN proteins, for example poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO:1)), poly(LS), poly(P), poly(QAGR (SEQ ID NO:2)), poly(RE), poly(SP), poly(VP), poly(FP), and/or poly(GK) RAN proteins, and combinations thereof, and methods of using such compositions.

Accordingly, in some aspects, the disclosure provides a method for preventing or treating a disease associated with RAN protein translation in a human subject in need thereof, the method comprising administering to the subject a therapeutically effective amount of a RAN protein vaccine, wherein administration of the RAN protein vaccine to the subject elicits production of one or more anti-RAN protein antibodies in the subject.

In some embodiments, the anti-RAN antibodies produced in the subject are one or more of: an anti-poly(Cysteine-Proline) [anti-poly(CP)]; anti-poly(Glycine-Proline) [anti-poly(GP)]; anti-poly(Glycine-Arginine) [anti-poly(GR)]; anti-poly(Glycine) [anti-poly(G)]; anti-poly(Alanine) [anti-poly(A)]; anti-poly(Serine) [anti-poly(S)]; anti-poly(Cysteine) [anti-poly(C)]; anti-poly(Glutamine) [anti-poly(Q)]; anti-poly(Glycine-Alanine) [anti-poly(GA)]; anti-poly(Glycine-Aspartate) [anti-poly(GD)]; anti-poly(Glycine-Glutamate) [anti-poly(GE)]; anti-poly(Glycine-Glutamine) [anti-poly(GQ)]; anti-poly(Glycine-Threonine) [anti-poly(GT)]; anti-poly(Leucine) [anti-poly(L)]; anti-poly(Leucine-Proline) [anti-poly(LP)]; anti-poly(Leucine-Proline-Alanine-Cysteine (SEQ ID NO:1)) [anti-poly(LPAC (SEQ ID NO: 1))]; anti-poly(Leucine-Serine) [anti-poly(LS)]; anti-poly(Proline) [anti-poly(P)]; anti-poly(Proline-Alanine) [anti-poly(PA)]; anti-poly(Proline-Arginine) [anti-poly(PR)]; anti-poly(Glutamine-Alanine-Glycine-Arginine (SEQ ID NO: 2)) [anti-poly(QAGR (SEQ ID NO: 2))]; anti-poly(Arginine-Glutamate) [anti-poly(RE)]; anti-poly(Serine-Proline) [anti-poly(SP)]; anti-poly(Valine-Proline) [anti-poly(VP)]; anti-poly(phenylalanine-proline) [anti-poly(FP)]; and/or anti-poly(glycine-lysine) [anti-poly(GK)] antibody, or any combination thereof.

In some embodiments, a disease or disorder associated with RAN proteins is amyotrophic lateral sclerosis (ALS), Huntington's disease (HD), Alzheimer's disease (AD), Fragile X Syndrome (FRAXA), Spinal Bulbar Muscular Atrophy (SBMA), Dentatorubropallidoluysian Atrophy (DRPLA), Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17), amyotrophic lateral sclerosis (ALS), Spinocerebellar ataxia type 36 (SCA36), Spinocerebellar ataxia type 29 (SCA29), Spinocerebellar ataxia type 10 (SCA10), myotonic dystrophy type 1 (DM1), myotonic dystrophy type 2 (DM2), or Fuch's Corneal Dystrophy (e.g., CTG181).

In some embodiments, the disease or disorder is ALS. In some embodiments, a subject having ALS is characterized by having one or more mutations in a C9Orf72 gene. In some embodiments, the disease or disorder is FTD. In some embodiments, a subject having FTD is characterized by having one or more mutations in a C9Orf72 gene. In some embodiments, the disease or disorder is HD. In some embodiments, a subject having HD is characterized by having one or more mutations in a Htt gene. In some embodiments, the disease or disorder is AD. In some embodiments, a subject having AD is characterized by having one or more mutations in an LRP8, CASP8, and/or GREB1 gene. In some embodiments, the disease or disorder is spinocerebellar ataxia type 36 (SCA36). In some embodiments, a subject having SCA36 is characterized by having one or more mutations in an SCA36 gene.

In some embodiments, a subject is characterized by RAN protein translation or the presence of RNA aggregates containing di-amino acid repeat (DPR)-encoding transcripts. In some embodiments, the RAN proteins being translated in a subject comprise one or more of the following: poly(GA), poly(GP), poly(GR), poly(PA), and/or poly(PR). In some embodiments, the poly(GA), poly(GP), poly(GR), poly(PA), and/or poly(PR) RAN proteins are expressed from a C9Orf72 expansion repeat of the subject. In some embodiments, the RAN proteins being translated in a subject comprise one or more of the following: poly(CP), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK). In some embodiments, the poly(CP), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK) RAN proteins are expressed from a C9Orf72, Htt, LRP8, CASP8, and/or GREB1 expansion repeat of the subject.

In some embodiments, a RAN protein vaccine comprises one or more peptide antigens. In some embodiments, the one or more peptide antigens comprise one or more immunogens. In some embodiments, the one or more peptide antigens comprise 1, 2, 3, 4, 5, 6, 7, 8, 9, or 10 immunogens. In some embodiments, the one or more peptide antigens target one or more RAN proteins, optionally wherein the one or more RAN proteins are one or more of poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK) RAN proteins.

In some embodiments, the one or more peptide antigens comprise one or more di-peptide repeat (DPR) peptide antigens. In some embodiments, each DPR peptide antigen comprises between 2 di-amino acid repeats and 150 di-amino acid repeats. In some embodiments, each DPR peptide antigen comprises between 10 di-amino acid repeats and 25 di-amino acid repeats. In some embodiments, each DPR peptide antigen comprises between 25 di-amino acid repeats and 50 di-amino acid repeats. In some embodiments, each DPR peptide antigen comprises between 50 di-amino acid repeats and 100 di-amino acid repeats. In some embodiments, each DPR peptide antigen comprises between 100 di-amino acid repeats and 150 di-amino acid repeats. In some embodiments, each di-peptide repeat antigen comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 di-amino acid repeats.

In some embodiments, each of the one or more DPR peptide antigens comprises a (GA)ₓ(SEQ ID NO: 4), (GP)ₓ (SEQ ID NO:5), (GR)ₓ (SEQ ID NO: 7), (PA)ₓ (SEQ ID NO: 8), or (PR)ₓ (SEQ ID NO: 6) di-amino acid repeat, wherein x represents the number of repeat units of the antigen. In some embodiments, each of the one or more DPR peptide antigens comprises a (CP)ₓ (SEQ ID NO:3), (A)ₓ (SEQ ID NO:9), (G)ₓ (SEQ ID NO:10), (S)ₓ (SEQ ID NO: 11), (C)ₓ (SEQ ID NO: 12), (Q)ₓ (SEQ ID NO: 13), (GD)ₓ (SEQ ID NO: 14), (GE)ₓ (SEQ ID NO: 15), (GQ)ₓ (SEQ ID NO:16), (GT)ₓ (SEQ ID NO:17), (L)ₓ (SEQ ID NO: 18), (LP)ₓ (SEQ ID NO: 19), (LPAC)_{X} (SEQ ID NO: 20), (LS)ₓ (SEQ ID NO: 21), (P)ₓ (SEQ ID NO: 22), (QAGR)_{X}(SEQ ID NO: 23), (RE)ₓ (SEQ ID NO: 24), (SP)ₓ (SEQ ID NO: 25), (VP)ₓ (SEQ ID NO: 26), (FP)ₓ (SEQ ID NO: 27), and/or (GK)ₓ (SEQ ID NO: 28) di-amino acid repeat, wherein x represents the number of repeat units of the antigen. In some embodiments, “x” is 5, 10, 15, 20, 25, 30, 35, 40, or more than 40 (e.g., 5, 10, 15, 20, 25, 30, 35, 40, or more than 40 repeat units).

In some embodiments, a DPR peptide antigen comprises (GA)₁₀(SEQ ID NO: 4), (GA)₁₅ (SEQ ID NO: 4), (GR)₂₅ (SEQ ID NO: 7), (GP)₁₀ (SEQ ID NO:5), (PR)₁₀ (SEQ ID NO: 6), or a combination thereof. In some embodiments, a DPR peptide antigen comprises [(GA)₁₅ (SEQ ID NO: 4)+(GR)₂₅ (SEQ ID NO: 7)+(PR)₁₀ (SEQ ID NO: 6)]. In some embodiments, a DPR peptide antigen comprises [(GA)₁₅ (SEQ ID NO: 4)+(GR)₂₅ (SEQ ID NO: 7)+(PR)₁₀ (SEQ ID NO: 6)+(GP)₁₀ (SEQ ID NO:5)].

In some embodiments, the one or more peptide antigens comprises a B-cell epitope.

In some embodiments, a RAN protein vaccine further comprises one or more additional immunogens. In some embodiments, the one or more immunogens comprise keyhole limpet hemocyanin (KLH), Blue Carrier Immunogenic protein (CCH), bovine serum albumin (BSA), ovalbumin (OVA), diphtheria toxin, measles virus fusion protein (MVF), hepatitis B virus surface antigen (HB-sAg), tetanus toxin (TT), pertussis toxin (PT), a T-cell helper epitope, or a portion of any of the foregoing.

In some embodiments, one or more additional immunogens are linked to a DPR peptide antigen via one or more linking molecules. In some embodiments, a linking molecule is selected from the group consisting of an amino acid, Lys-, Gly-, Lys-Lys-Lys-, (α, ε-N)Lys, and ε-N-Lys-Lys-Lys-Lys (SEQ ID NO: 29).

In some embodiments, a RAN protein vaccine comprises a formula set forth in Table 1.

In some embodiments, each of the one or more anti-RAN protein antibodies produced by a subject bind to a poly(GP), poly(GA), poly(GR), or poly(PR) di-amino acid repeat-containing RAN protein. In some embodiments, each of the one or more anti-RAN protein antibodies produced by a subject bind to a poly(CP), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK) di-amino acid repeat-containing RAN protein.

In some embodiments, the anti-RAN protein antibodies are one or more of an anti-poly(CP), anti-poly(GA), anti-poly(GP), anti-poly(PR), anti-poly(GR), anti-poly(PA), anti-poly(A), anti-poly(G), anti-poly(S); anti-poly(C); anti-poly(Q); anti-poly(GD), anti-poly(GE), anti-poly(GQ), anti-poly(GT), anti-poly(L), anti-poly(LP), anti-poly(LPAC (SEQ ID NO: 1)), anti-poly(LS), anti-poly(P), anti-poly(QAGR (SEQ ID NO: 2)), anti-poly(RE), anti-poly(SP), anti-poly(VP), anti-poly(FP), or anti-poly(GK) antibody.

In some embodiments, a subject is a mammal. In some embodiments, a subject is a human.

In some embodiments, a subject is administered one or more additional doses of a RAN protein vaccine.

In some embodiments, administration of a RAN protein vaccine reduces RAN protein expression relative to the level of RAN protein expression present in the subject prior to administration of the RAN protein vaccine. In some embodiments, administration of a RAN protein vaccine reduces RAN protein expression by one-fold, two-fold, three-fold, four-fold, five-fold, six-fold, seven-fold, eight-fold, nine-fold, ten-fold, fifteen-fold, twenty-fold, thirty-fold, forty-fold, fifty-fold, sixty-fold, seventy-fold, eighty-fold, ninety-fold, one hundred-fold, or more than one-hundred fold relative to the level of RAN protein expression present in the subject prior to administration of the RAN protein vaccine. In some embodiments, administration of a RAN protein vaccine reduces RAN protein expression by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage contained therein, relative to the level of RAN protein expression present in the subject prior to administration of the RAN protein vaccine.

In some embodiments, administration of a RAN protein vaccine reduces RAN protein aggregation relative to the level of RAN protein aggregation present in the subject prior to administration of the RAN protein vaccine. In some embodiments, administration of a RAN protein vaccine reduces RAN protein aggregation by one-fold, two-fold, three-fold, four-fold, five-fold, six-fold, seven-fold, eight-fold, nine-fold, ten-fold, fifteen-fold, twenty-fold, thirty-fold, forty-fold, fifty-fold, sixty-fold, seventy-fold, eighty-fold, ninety-fold, one hundred-fold, or more than one-hundred fold relative to the level of RAN protein aggregation present in the subject prior to administration of the RAN protein vaccine. In some embodiments, administration of a RAN protein vaccine reduces RAN protein aggregation by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage contained therein, relative to the level of RAN protein aggregation present in the subject prior to administration of the RAN protein vaccine.

In some embodiments, the RAN protein vaccine further comprises one or more adjuvants. In some embodiments, the one or more adjuvants comprise any one or more of: immune targeting adjuvants; immunomodulating adjuvants; incomplete Freund's adjuvant; aluminum phosphate; aluminum hydroxide; alum; Stimulon® QS-21; MPL®; interleukin-12; an oil formulation; a polymer; a micelle forming adjuvant; a saponin; an immunostimulating complex matrix (ISCOM matrix); a particle; DDA; DNA adjuvants; an encapsulating adjuvant; flagellin adjuvants; alhydrogel (Al(OH)₃); CpG1; CpG3; and/or adjuphos (AlPO₄).

In some embodiments, the RAN protein vaccine is delivered to the subject in a composition comprising an exosome. In some embodiments, the RAN protein vaccine is delivered to the subject in a composition comprising a nanoparticle. In some embodiments, the nanoparticle is a lipid nanoparticle.

In some embodiments, the RAN protein vaccine is an mRNA vaccine.

### BRIEF DESCRIPTION OF DRAWINGS

FIG. 1 shows a schematic depicting a study design for testing of peptide-based anti-RAN protein vaccines as performed in the Example.

FIG. 2 shows a schematic of di-amino acid repeat RAN protein expression constructs. Constructs were expressed in mammalian cells for production of RAN proteins (e.g., poly(GA), poly(GP), poly(GR), poly(PR), etc.), ranging in lengths from 30 to 60 to 120 di-amino acid repeats. Expression of di-amino acid repeat coding sequences was driven by a CMV promoter. Constructs also contained a V5 epitope tag or FLAG tag. Constructs having alternative codon usage (“Alt codon”) were also used.

FIGS. 3A-3D show representative Western blots indicating sera (e.g., sera containing anti-RAN protein antibodies) from guinea pigs exposed to the noted peptide-based anti-RAN protein vaccine (e.g., GA₁₀ (SEQ ID NO: 4), GP₁₀ (SEQ ID NO: 5), GR₂₅ (SEQ ID NO: 7), PR₁₀ (SEQ ID NO: 6)) were able to bind RAN proteins expressed by the constructs described in FIG. 2 in vitro. FIG. 3A shows a representative Western blot for sera from animals injected with a GA₁₀ (SEQ ID NO: 4) peptide vaccine. FIG. 3B shows a representative Western blot for sera from animals injected with a GP₁₀ (SEQ ID NO: 5) peptide vaccine. FIG. 3C shows a representative Western blot for sera from animals injected with a GR₂₅ (SEQ ID NO: 7) peptide vaccine. FIG. 3D shows a representative Western blot for sera from animals injected with a PR₁₀ (SEQ ID NO: 6) peptide vaccine.

FIG. 4 shows a summary of in vitro protein assay results.

FIG. 5 shows representative immunohistochemistry data indicating that anti-RAN protein antibodies in sera obtained from guinea pigs exposed to a peptide-based anti-RAN protein vaccine recognize RAN proteins in situ. Top and bottom left: anti-GA antibody in C9+ mouse tissue and control mouse tissue, respectively; Top and bottom right: GA₂₅ (SEQ ID NO: 4) sera in C9+ mouse tissue and control mouse tissue, respectively.

FIG. 6 shows a schematic depicting a study design for in vivo mouse vaccination studies.

FIG. 7 shows a summary of vaccine groups for the mouse study described in FIG. 6.

FIGS. 8A-8B show representative immunohistochemistry data. FIG. 8A shows poly(GA) RAN protein aggregates in the retrosplenial cortex (RSC) of C9+ mice injected with the GA₁₅ (SEQ ID NO: 4) vaccine or adjuvant. FIG. 8B shows mouse anti-RAN protein antibodies are produced in GA₁₅ (SEQ ID NO: 4) vaccinated mice and form puncta in the RSC of C9+ mice, but not no-treatment (NT) mice.

FIGS. 9A-9B show representative fluorescence microscopy data indicating anti-RAN protein antibodies produced by vaccinated mice co-localized with GA aggregates detected in the brain with anti-rabbit antibodies. FIG. 9A shows data for a C9+ mouse vaccinated with GA₁₅ (SEQ ID NO: 4) vaccine. FIG. 9B shows data for a C9+ mouse vaccinated with a combination (GA₁₅ (SEQ ID NO: 4)+GR₂₅ (SEQ ID NO: 7)+PR₁₀ (SEQ ID NO: 6)+GP₁₀(SEQ ID NO: 5)) vaccine.

FIG. 10 shows GA aggregates were reduced in mice treated with GA₁₅ (SEQ ID NO: 4) vaccine. 6 μm brain sections from C9(+) mice treated with either the GA₁₅ (SEQ ID NO: 4) vaccine (n=4) or adjuvant control (n=3) were stained by immunohistochemistry for the presence of GA aggregates using an anti-GA antibody and a hematoxylin counterstain. Total GA aggregates in the retrosplenial cortex of C9(+) mice treated with GA₁₅ (SEQ ID NO: 4) vaccine (n=4) or adjuvant control (n=3). Data are presented as GA aggregates/total cells (n=3 animals/group).

### DETAILED DESCRIPTION

Aspects of the disclosure relate to compositions and methods for eliciting (or enhancing) anti-RAN protein antibody expression or production in a subject. The disclosure is based, in part, on compositions for vaccinating a subject to produce antibodies against certain RAN proteins, for example poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and poly(GK) RAN proteins, and combinations thereof. In some embodiments, methods and compositions described by this disclosure are useful for preventing or treating (e.g., delaying the onset or progression) of certain diseases and disorders associated with RAN proteins. In some embodiments, methods and compositions described by the disclosure are useful for diagnosing certain diseases and disorders associated with RAN proteins. In some embodiments, methods and compositions described by the disclosure are useful for monitoring the progression of certain diseases and disorders associated with RAN proteins.

In some embodiments, a disease or disorder associated with RAN proteins is any one of: amyotrophic lateral sclerosis (ALS), frontotemporal dementia (FTD), myotonic dystrophy type 1 (DM1), myotonic dystrophy type 2 (DM2), spinocerebellar ataxia types 1, 2, 3, 6, 7, 8, 10, 12, 17, 31, and 36, spinal bulbar muscular atrophy, dentatorubral-pallidoluysian atrophy (DRPLA), Huntington's disease (HD), Alzheimer's disease (AD), Fragile X Tremor Ataxia Syndrome (FXTAS), Fuch's endothelial corneal dystrophy (FECD), Huntington's disease-like 2 syndrome (HDL2), Fragile X syndrome (FXS), disorders related to 7p11.2 folate-sensitive fragile site FRA7A, disorders related to folate-sensitive fragile site 2q11.2FRA2A, or Fragile XE syndrome (FRAXE).

### RAN Proteins

In some aspects, the disclosure relates to compositions and methods for producing (or enhancing production of) anti-RAN protein antibodies in a subject. A “RAN protein (repeat-associated non-ATG translated protein)” is a polypeptide translated from mRNA sequence carrying a nucleotidic expansion in the absence of an AUG initiation codon. Generally, RAN proteins comprise expansion repeats of a single amino acid, di-amino acid, tri-amino acid, or quad-amino acid (e.g., tetra-amino acid), termed poly-amino acid repeats. The amino acid repeats may be homopolymeric amino acid repeats, di-amino acid repeats, tri-amino acid repeats, tetra-amino acid repeats, or penta-amino acid repeats. Generally, a RAN protein may comprise between about 2 and about 10,000 amino acid repeats. In some embodiments, a RAN protein comprises between 20 and 100 amino acid repeats, 50 and 200 amino acid repeats, 100 and 500 amino acid repeats, 400 and 800 amino acid repeats, 700 and 1000 amino acid repeats, 800 and 1500 amino acid repeats, etc. In some embodiments, a RAN protein comprises a poly-amino acid repeat that is between 10 and 500 amino acid residues in length. In some embodiments, a RAN protein comprises a poly-amino acid repeat that is between 20 and 300 amino acid residues in length. In some embodiments, a RAN protein comprises a poly-amino acid repeat that is between 30 and 200 amino acid residues in length. In some embodiments, a RAN protein comprises a poly-amino acid repeat that is between 40 and 100 amino acid residues in length. In some embodiments, a RAN protein comprises a poly-amino acid repeat that is between 50 and 90 amino acid residues in length. In some embodiments, a RAN protein comprises a poly-amino acid repeat that is between 60 and 80 amino acid residues in length. In some embodiments, a RAN protein comprises a poly-amino acid repeat that is at least 20, at least 25, at least 30, at least 40, at least 50, at least 60, at least 70, at least 80, at least 90, at least 100, or at least 200 amino acid residues in length. In some embodiments, a RAN protein has a poly-amino acid repeat more than 200 amino acid residues (e.g., 500, 1000, 5000, 10,000, etc.) in length.

RAN protein-encoding sequences can be found in a subject's genome (e.g., a human subject's genome) at multiple loci, including, but not limited to, open reading frame 72 of chromosome 9 (C9orf72), open reading frame 80 of chromosome 2 (C2orf80), LRP8, CASP8, CRNDE, EXOC6B, SV2B, PPML1, ADARB2, GREB1, and MSMO1. The protein associated with C9orf72 is currently poorly characterized but known to be abundant in neurons, especially in the cerebral cortex and motor neurons. C9orf72 protein is believed to localized in presynaptic termini. C9orf72 protein likely impacts transcription, translation, and intra-cellular localization of RNA. The C9orf72 gene contains a GGGGCC repeat (a hexanucleotide repeat), which occurs in variable repeat numbers.

In the context of ALS/FTD, which results from a repeat expansion of the hexanucleotide sequence GGGGCC in the C9Orf72 gene, the following di-amino acid repeat-containing RAN proteins have been identified: poly-(Gly-Ala), poly-(Gly-Pro), poly-(Gly-Arg), poly-(Pro-Ala), or poly-(Pro-Arg), also referred to as poly(GA), poly(GP), poly(GR), poly(PA), and poly(PR), respectively. ALS/FTD RAN proteins are generally described, for example, in International PCT Application PCT/US2014/022670, filed on Mar. 10, 2014, published as WO2014/159247, and U.S. application Ser. No. 14/775,278, filed on Sep. 11, 2015, published as US2016/0025747, the entire contents of each of which are incorporated by reference herein.

In the context of SCA36, which results from a repeat expansion of the hexanucleotide sequence TGGGCC in the SCA36 gene, the following di-amino acid repeat-containing RAN proteins have been identified: poly(GP) and poly(PR).

In the context of Huntington's disease (HD), RAN protein translation is caused by a CAG·CTG expansion in the Htt gene, which results in translation of RAN proteins polyAlanine, polySerine, polyLeucine, and polyCysteine (polyAla, polySer, polyLeu and polyCys), in addition to poly-Glutamine (polyGln or polyQ).

In the context of SCA8 and DM1, RAN protein translation is caused by a CTG·CAG repeat expansion. The SCA8 expansion mutation is bidirectionally transcribed and produces both CUG (ATXN8OS) and CAG (ATXN8) expansion RNAs, which are expressed in opposite directions across the expansion mutation. The CUG expansion transcripts form RNA foci, and the expanded CAG ATXN8 transcript expressed in the opposite direction produces a nearly pure polyGln protein from an unusually short ORF that contains an AUG-initiation codon directly upstream of the CAG repeat. This results in translation of the RAN proteins polyAlanine, polySerine, polyLeucine, and polyCysteine (polyAla, polySer, polyLeu and polyCys), in addition to poly-Glutamine (polyGln or polyQ).

In the context of Fragile X Syndrome (FXS) and Fragile X-associated tremor/ataxia syndrome (FXTAS), RAN protein translation is caused by a CGG·CCG expansion. Expansion of the CGG repeat tract in the 5′ untranslated region (UTR) of the FMR1 gene causes two distinct diseases, depending on the length of the repeat. Larger expansions (>200 repeats) cause transcriptional silencing of the FMR1 gene resulting in FXS. In contrast, shorter alleles within the premutation range (55-200 repeats) cause FXTAS, a late-onset neurodegenerative disorder. This results in translation of RAN proteins polyGly, polyPro, polyArg, and polyAla.

In the context of DM2, RAN protein translation is caused by an intronic CCTG·CAGG expansion mutation located in the cellular nucleic acid binding protein (CNBP) gene, which produces tetrapeptide expansion proteins in both the sense (poly(leucine-proline-alanine-cysteine [LPAC])) and antisense (poly(glutamine-alanine-glycine-arginine [QAGR])) directions.

In the context of SCA31, RAN protein translation is caused by the accumulation of a UGGAA expansion-encoded Trp-Asn-Gly-Met-Glu (SEQ ID NO: 30) pentapeptide repeat protein (PPR).

Other examples of RAN proteins may include poly(CP), poly(GD), poly(GE), poly(GQ), poly(GT), poly(LP), poly(LPAC (SEQ ID NO: 1)), poly(LS), poly(P), poly(QAGR (SEQ ID NO: 2)), poly(RE), poly(SP), poly(VP), poly(FP), or poly(GK) RAN proteins, or any combination thereof. Examples of additional RAN proteins and methods of identifying RAN proteins are described, for example, in International PCT Application PCT/US2020/040725, filed on Jul. 2, 2020, the entire contents of which are incorporated herein by reference.

It should be appreciated that peptide vaccines may be configured to elicit anti-RAN protein antibodies targeting polyAla, polySer, polyLeu, polyCys, and polyGln using methods described by the disclosure (e.g., by substituting a homopolymeric amino acid repeat peptide antigen for a di-amino acid repeat peptide antigen in a RAN protein vaccine). Other examples of RAN proteins may include poly(CP), poly(GD), poly(GE), poly(GQ), poly(GT), poly(LP), poly(LPAC (SEQ ID NO: 1)), poly(LS), poly(P), poly(QAGR (SEQ ID NO: 2)), poly(RE), poly(SP), poly(VP), poly(FP), poly(GK), or any combination thereof.

In some embodiments, the RAN protein is encoded by a gene associated with Huntington's disease (HD, HDL2), Alzheimer's disease (AD), Fragile X Syndrome (FRAXA), Spinal Bulbar Muscular Atrophy (SBMA), Dentatorubropallidoluysian Atrophy (DRPLA), Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17), amyotrophic lateral sclerosis (ALS), Spinocerebellar ataxia type 36 (SCA36), Spinocerebellar ataxia type 29 (SCA29), Spinocerebellar ataxia type 10 (SCA10), myotonic dystrophy type 1 (DM1), myotonic dystrophy type 2 (DM2), or Fuch's Corneal Dystrophy (e.g., CTG181).

### Subjects and Biological Samples

Aspects of the disclosure relate to compositions and methods for eliciting (or enhancing) anti-RAN protein antibody expression or production in a subject. A subject can be a mammal (e.g., human, mouse, rat, dog, cat, guinea pig, pig, etc.). In some embodiments, a subject is a mammalian subject. In some embodiments, the subject is a human. In some embodiments, a subject is a mouse. In some embodiments, a subject is a C9-BAC mouse. The C9-BAC mouse model of ALS is described, for example, in International PCT Application PCT/US2014/022670, filed on Mar. 10, 2014, published as WO2014/159247, and Liu et al., (2016) Neuron 90(3):521-34, the entire contents of each of which are incorporated herein by reference.

In some embodiments, a subject is characterized by a GGGGCC (e.g., G₄C2) hexanucleotide sequence repeat expansion in the C9Orf72 gene (e.g., a human C9Orf72 gene or a gene, such as a mouse gene, corresponding to human C9Orf72 gene). In some embodiments, a human C9Orf72 gene comprises or consists of the sequence set forth in any one of NCBI Reference Sequence Numbers NM_145005.6, NM_018325.4, and NM_001256054.2. In some embodiments, a subject is characterized by a TGGGCC hexanucleotide sequence repeat expansion in the SCA36 gene (e.g., a human SCA36 gene or a gene, such as a mouse gene, corresponding to human SCA36 gene). In some embodiments, a human SCA36 gene comprises or consists of the sequence set forth in any one of NCBI Reference Sequence Numbers NM_006392.3, NR_027700.2, and NR_145428.1. In some embodiments, a subject has been determined to have a hexanucleotide sequence repeat expansion (e.g., a GGGGCC (e.g., G₄C₂) repeat expansion in C9Orf72 or a TGGGCC repeat expansion in SCA36) by a genetic assay (e.g., a DNA-based assay, for example a sequencing assay).

In some embodiments, a subject comprises at least 50, at least 100, at least 200, at least 500, at least 1000, or at least 5000 GGGGCC repeat expansions (e.g., repeat expansions of C9Orf72). In some embodiments, a subject comprises at least 50, at least 100, at least 200, at least 500, at least 1000, or at least 5000 TGGGCC repeat expansions (e.g., repeat expansions of SCA36).

In some embodiments, a subject is characterized by RAN protein translation or the presence of RNA aggregates containing di-amino acid repeat (DPR)-encoding transcripts. In some embodiments, the RAN proteins being translated in a subject comprise one or more of the following: poly(GA), poly(GP), poly(GR), poly(PA), or poly(PR). In some embodiments, the RAN proteins being translated in a subject comprise one or more of the following: poly(CP), poly(GD), poly(GE), poly(GQ), poly(GT), poly(LP), poly(LPAC (SEQ ID NO: 1)), poly(LS), poly(P), poly(QAGR (SEQ ID NO: 2)), poly(RE), poly(SP), poly(VP), poly(FP), poly(GK), or any combination thereof. In some embodiments, the poly(GA), poly(GP), poly(GR), poly(PA), poly(PR) RAN proteins are expressed from a C9Orf72, Htt, SCA36, LRP8, CASP8, and/or GREB1 expansion repeat of the subject. In some embodiments, the poly(CP), poly(GD), poly(GE), poly(GQ), poly(GT), poly(LP), poly(LPAC (SEQ ID NO: 1)), poly(LS), poly(P), poly(QAGR (SEQ ID NO: 2)), poly(RE), poly(SP), poly(VP), poly(FP), poly(GK) proteins are expressed from a C9Orf72, Htt, SCA36, LRP8, CASP8, and/or GREB1 expansion repeat of the subject.

A “subject having or suspected of having ALS and/or FTD” can be a subject that is known or determined to have more than 30 GGGGCC repeats in the C9Orf72 gene, or a subject exhibiting signs and symptoms of ALS/FTD, including but not limited to: motor dysfunction (e.g., spasticity), muscle atrophy, and/or neuropsychiatric manifestations (e.g., compulsive behavior, apathy, anxiety. In some embodiments, a subject having ALS is characterized by having one or more mutations in a C9Orf72 gene.

A “subject having or suspected of having Alzheimer's disease” can be a subject exhibiting one or more signs and symptoms of AD, including but not limited to: memory deficit (e.g., short term memory loss), confusion, deficiencies of executive functions (e.g., attention, planning, flexibility, abstract thinking, etc.), loss of speech, degeneration or loss of motor skills, etc., or a subject having or being identified as having one or more genetic mutations associated with AD, for example mutations in specific genes including apolipoprotein (APP), presenillin genes (PSEN1 and PSEN2), or tau protein. In some embodiments, a subject having or suspected of having AD is characterized by the accumulation of β-amyloid (Aβ) peptides and hyper-phosphorylated tau protein throughout brain tissue of the subject. In some embodiments, a subject has been diagnosed as having AD by a medical professional, according to the NINCDS-ADRDA Alzheimer's Criteria, as described by McKhann et al., (1984) “Clinical diagnosis of Alzheimer's disease: report of the NINCDS-ADRDA Work Group under the auspices of Department of Health and Human Services Task Force on Alzheimer's Disease”. Neurology, 34 (7): 939-44.

A “subject having or suspected of having myotonic dystrophy” (e.g., myotonic dystrophy type 1 (DM1) or myotonic dystrophy type 2 (DM2)) can be a subject exhibiting one or more signs or symptoms of DM1 and/or DM2, including but not limited to: delayed muscle relaxation, muscle weakness, prolonged involuntary muscle contraction, or loss of muscle; and/or abnormal heart rhythm, cataracts, or difficulty swallowing.

A “subject having or suspected of having spinocerebellar ataxia” (e.g., spinocerebellar ataxia types 1, 2, 3, 6, 7, 8, 10, 12, 17, 31, or 36) can be a subject exhibiting one or more signs or symptoms of spinocerebellar ataxia, including but not limited to: speech and swallowing difficulties, muscle stiffness (e.g., spasticity), weakness in the muscles that control eye movement (e.g., ophthalmoplegia), rapid, involuntary eye movements (e.g., nystagmus), uncoordinated movement and poor balance (e.g., ataxia), muscle wasting, slow eye movement, dementia, uncontrolled muscle tensing (e.g., dystonia), rigidity, tremors, bulging eyes, double vision, loss of coordination in arms, progressive vision loss, blindness, changes in sensation or reflexes, truncal instability, hyperactive tendon reflexes, scanning dysarthria characterized by a drawn-out slowness of speech, cerebellar ataxia, unsteady gait, upper-limb ataxia, dysphagia, gait dysfunction, extrapyramidal features, pyramidal weakness, cognitive and behavioral disturbances, chorea, psychiatric disturbances, sensorineural hearing impairment, impaired vibratory sensation, rapid eye movements (e.g., saccades), trouble moving the eyes side-to-side (e.g., oculomotor apraxia), and/or droopy eyelids (e.g., ptosis).

A “subject having or suspected of having spinal bulbar muscular atrophy” can be a subject exhibiting one or more signs or symptoms of spinal bulbar muscular atrophy, including but not limited to: speech impairment, difficulty chewing and swallowing, impaired sleep, difficulty breathing, facial muscle weakness, difficulty conveying emotion, weakness and atrophy of the arm and leg muscles, twitching and cramping of muscles, enlarged breasts (in male subjects), reduced fertility and atrophy (e.g., shrinkage) of the testicles, abnormal processing of male hormones, muscle wasting, and/or difficulty walking.

A subject “having or suspected of having dentatorubral-pallidoluysian atrophy (DRPLA)” can be a subject exhibiting one or more signs or symptoms of DRPLA, including but not limited to: ataxia, uncontrollable movements of the limbs (e.g., choreoathetosis), psychiatric symptoms (e.g., delusions), and/or deterioration of intellectual function (e.g., dementia).

A subject “having or suspected of having Huntington's disease (HD)” can be a subject exhibiting one or more signs or symptoms of HD, including but not limited to: abnormality walking, increased muscle activity, involuntary movements, problems with coordination, loss of muscle, muscle spasms, amnesia, delusion, lack of concentration, mental confusion, slowness in activity, difficulty thinking and understanding, compulsive behavior, fidgeting, irritability, lack of restraint, delirium, depression, hallucination, paranoia, anxiety, apathy, mood swings, difficulty speaking, memory loss, tremor, and/or weight loss.

A subject “having or suspected of having a Fragile X disorder” (e.g. Fragile X syndrome (FXS), Fragile X Tremor Ataxia Syndrome (FXTAS), or Fragile XE syndrome (FRAXE)) can be a subject exhibiting one or more signs or symptoms of FXS, FXTAS, and/or FRAXE, including but not limited to: aggression, hyperactivity, impulsivity, nonsense word repetition, repetitive movements, self-harm, persistent repetition of words or actions, learning disability or speech delay (e.g., in a child), flaccid muscles or problems with coordination, large ears, speech impairment, anxiety, double jointed limbs, enlarged head, enlarged testicles, flat feet, lazy eye, long thin face, prominent jaw, scoliosis, single line on palm, sleep disorder, sunken chest, tremor, neuropathy, numbness/tingling of the extremities, mood instability, irritability, explosive outbursts, personality changes, cognitive decline (including loss of skills such as math, reading, etc.), autonomic functioning problems (such as impotence and loss of bladder or bowel functions), delayed speech, poor writing skills, hyperactivity, and/or a short attention span.

A subject “having or suspected of having Huntington's disease-like 2 syndrome (HDL2)” can be a subject exhibiting one or more signs or symptoms of HDL2, including but not limited to: progressive movement disorder (e.g., parkinsonism, chorea), cognitive and emotional decline (e.g., dementia, psychiatric disturbances), epileptic seizure(s), and/or any other signs or symptoms associated with HD.

A subject “having or suspected of having Fuchs' endothelial corneal dystrophy (FECD)” can be a subject exhibiting one or more signs or symptoms of FECD, including but not limited to: blurred or cloudy vision (e.g., a general lack of clarity of vision), fluctuation in vision (e.g., worse symptoms in the morning after awakening and gradually improving during the day), permanent vision impairment, glare, seeing halos around lights, and/or pain or grittiness from tiny blisters on the surface of cornea.

A subject “having or suspected of having a disorder related to 7p11.2 folate-sensitive fragile site FRA7A” can be a subject exhibiting one or more symptoms of a disorder related to 7p11.2 folate-sensitive fragile site FRA7A, including but not limited to: inappropriate social interaction, poor eye contact, compulsive behavior, impulsivity, repetitive movements, self-harm, persistent repetition of words or actions, learning disability or speech delay (e.g., in a child), intense interest in a limited number of things, problem paying attention, unaware of others' emotions or depression, anxiety, change in voice, sensitivity to sound, and/or tic.

A subject “having or suspected of having a disorder related to folate-sensitive fragile site 2q11.2 FRA2A” can be a subject exhibiting one or more symptoms of a disorder related to folate-sensitive fragile site 2q11.2 FRA2A, including but not limited to: social isolation, disorganized behavior, aggression, agitation, compulsive behavior, excitability, hostility, repetitive movements, self-harm, lack of restraint, thought disorder, delusion, amnesia, belief that an ordinary event has special and personal meaning, belief that thoughts aren't one's own, disorientation, mental confusion, slowness in activity, false belief of superiority, anger, anxiety, apathy, feeling detached from self, general discontent, loss of interest or pleasure in activities, elevated mood, inappropriate emotional response, hallucination, paranoia, hearing voices, depression, fear, persecutory delusion, religious delusion, circumstantial speech, incoherent speech, rapid and frenzied speaking, speech disorder, fatigue, impaired motor coordination, lack of emotional response, memory loss, and/or other intellectual disabilities.

### Anti-RAN Protein Antibodies

The disclosure is based, in part, on administration of certain RAN protein antigens (e.g., di-amino acid repeat (DPR) peptide antigens) to initiate or enhance anti-RAN protein antibody expression or production in a subject (e.g., peptide vaccines and immunogen compositions that elicit an anti-RAN protein immune response in the subject).

An anti-RAN protein antibody can be a polyclonal antibody or a monoclonal antibody. Typically, polyclonal antibodies are produced by inoculation of a suitable mammal, such as a human, mouse, rabbit, guinea pig, or goat. Larger mammals are often preferred as the amount of serum that can be collected is greater. An antigen is injected into the mammal. This induces the B-lymphocytes to produce IgG immunoglobulins specific for the antigen. Monoclonal antibodies are generally produced by a single cell line (e.g., a hybridoma cell line). In some embodiments, a polyclonal IgG is purified from a mammal's serum.

In some embodiments, for example in the case of administration of a vaccine against a RAN protein-associated disease or disorder, the polyclonal antibodies may freely circulate in the serum of the animal. In some embodiments, the anti-RAN protein antibodies produced by a subject that has been administered a composition of the disclosure are autoantibodies (e.g., antibodies to RAN proteins produced by the subject's own cells and not as a result of administration of a therapeutic antibody or gene therapy vector encoding an anti-RAN protein antibody).

In some embodiments, an anti-RAN protein antibody is an anti-poly(CP), anti-poly(GA), anti-poly(GP), anti-poly(PR), anti-poly(GR), anti-poly(PA), anti-poly(A), anti-poly(G), anti-poly(S), anti-poly(C), anti-poly(Q), anti-poly(GD), anti-poly(GE), anti-poly(GQ), anti-poly(GT), anti-poly(L), anti-poly(LP), anti-poly(LPAC (SEQ ID NO: 1)), anti-poly(LS), anti-poly(P), anti-poly(QAGR (SEQ ID NO: 2)), anti-poly(RE), anti-poly(SP), anti-poly(VP), anti-poly(FP), or anti-poly(GK) antibody, or any combination thereof. An anti-RAN protein antibody may bind to an extracellular RAN protein, an intracellular RAN protein, or both extracellular and intracellular RAN proteins.

In some embodiments, an anti-RAN protein antibody targets (e.g., specifically binds to) the amino acid repeat region (e.g., a di-amino acid repeat of a RAN protein, a homopolymeric repeat of a RAN protein, etc.) of one or more RAN proteins selected from: poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK). In some embodiments, the target amino acid repeat region of the one or more RAN proteins comprises at least 20, at least 25, at least 30, at least 40, at least 50, at least 60, at least 70, at least 80, at least 90, at least 100, or at least 200 amino acid residues. In some embodiments, the target amino acid repeat region of the one or more RAN proteins comprises more than 200 amino acid residues (e.g., 500, 1000, 5000, 10000, etc.). In some embodiments, the target amino acid repeat region of the one or more RAN proteins is between 10 and 500 amino acids in length. In some embodiments, the target amino acid repeat region of the one or more RAN proteins is between 20 and 300 amino acids in length. In some embodiments, the target amino acid repeat region of the one or more RAN proteins is between 30 and 200 amino acids in length. In some embodiments, the target amino acid repeat region of the one or more RAN proteins is between 40 and 100 amino acids in length. In some embodiments, the target amino acid repeat region of the one or more RAN proteins is between 50 and 90 amino acids in length. In some embodiments, the target amino acid repeat region of the one or more RAN proteins is between 60 and 80 amino acids in length.

In some embodiments, an anti-RAN antibody targets (e.g., specifically binds to) any portion of a RAN protein that does not comprise the poly-amino acid repeat, for example the C-terminus of a RAN protein (e.g., the C-terminus of a poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK) RAN protein). Examples of anti-RAN antibodies targeting the C-terminus of RAN protein are disclosed, for example, in U.S. Publication No. 2013/0115603, the entire contents of which are incorporated herein by reference.

A “C-terminal portion” or “C-terminus” of a RAN protein comprises the amino acid sequence encoded by a nucleotide sequence downstream of the poly-amino acid repeat region within the intron of a gene (e.g., C9Orf72, HTT, DM1, SCA36, LRP8, CASP8, GREB1, etc.) for the sense transcript or a nucleotide sequence downstream of the poly-amino acid repeat region within the intron of a gene (e.g., C9Orf72, HTT, DM1, SCA36, LRP8, CASP8, GREB1, etc.) for the anti-sense transcript.

In some embodiments, the C-terminal portion of a RAN protein comprises one or more contiguous amino acids in a sequence which begins at the amino acid immediately following the poly-amino acid repeat portion of the RAN protein and which is encoded by the sense transcript of the gene (e.g., C9Orf72, HTT, DM1, SCA36, LRP8, CASP8, GREB1, etc.). In some embodiments, the C-terminal portion of a RAN protein comprises one or more contiguous amino acids in a sequence which begins at the amino acid immediately following the poly-amino acid repeat portion of the RAN protein and which is encoded by the antisense transcript of the gene (e.g., C9Orf72, HTT, DM1, SCA36, LRP8, CASP8, GREB1, etc.).

In some embodiments, the C-terminal portion of a RAN protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more than 75 contiguous amino acids in a sequence which begins at the amino acid immediately following the poly-amino acid repeat portion of the RAN protein. In some embodiments, the C-terminal portion of a RAN protein comprises 1-5, 3-10, 5-15, 8-20, 10-25, 13-30, 15-35, 18-40, 20-45, 23-50, 25-55, 28-60, 30-65, 33-70, 35-75, or more than 75 contiguous amino acids in a sequence which begins at the amino acid immediately following the poly-amino acid repeat portion of the RAN protein.

### RAN Protein Vaccines and Immunogens

A RAN protein vaccine generally comprises one or more peptide antigens that elicit an immune response (e.g., production of specific antibodies in the subject) to a RAN protein. In some embodiments, one or more of the peptide antigens is a B-cell epitope. In some embodiments, the peptide antigen targets (e.g., comprises an amino acid sequence encoding) one or more of the RAN proteins: poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK).

In some embodiments, administration of a RAN protein vaccine reduces RAN protein expression relative to the level of RAN protein expression present in the subject prior to administration of the RAN protein vaccine. In some embodiments, administration of a RAN protein vaccine reduces RAN protein expression by one-fold, two-fold, three-fold, four-fold, five-fold, six-fold, seven fold-eight-fold, nine-fold, ten-fold, fifteen-fold, twenty-fold, thirty-fold, forty-fold, fifty-fold, sixty-fold, seventy-fold, eighty-fold, ninety-fold, one hundred-fold, or more than one-hundred fold relative to the level of RAN protein expression present in the subject prior to administration of the RAN protein vaccine. In some embodiments, administration of a RAN protein vaccine reduces RAN protein expression by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage contained therein relative to the level of RAN protein expression present in the subject prior to administration of the RAN protein vaccine.

In some embodiments, administration of a RAN protein vaccine reduces RAN protein aggregation relative to the level of RAN protein aggregation present in the subject prior to administration of the RAN protein vaccine. In some embodiments, administration of a RAN protein vaccine reduces RAN protein aggregation by one-fold, two-fold, three-fold, four-fold, five-fold, six-fold, seven fold-eight-fold, nine-fold, ten-fold, fifteen-fold, twenty-fold, thirty-fold, forty-fold, fifty-fold, sixty-fold, seventy-fold, eighty-fold, ninety-fold, one hundred-fold, or more than one-hundred fold relative to the level of RAN protein aggregation present in the subject prior to administration of the RAN protein vaccine. In some embodiments, administration of a RAN protein vaccine reduces RAN protein aggregation by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or any percentage contained therein relative to the level of RAN protein aggregation present in the subject prior to administration of the RAN protein vaccine.

In some embodiments, a peptide antigen is a di-peptide repeat (DPR) peptide antigen. In some embodiments, a DPR peptide antigen comprises a (GA)ₓ (SEQ ID NO: 4), (GP)ₓ (SEQ ID NO: 5), (GR)ₓ (SEQ ID NO: 7), (PA)ₓ (SEQ ID NO: 8), or (PR)ₓ (SEQ ID NO: 6) di-amino acid repeat, wherein x represents the number of repeat units of the antigen. In some embodiments, a DPR peptide antigen comprises a (CP)ₓ (SEQ ID NO:3), (A)ₓ (SEQ ID NO:9), (G)ₓ (SEQ ID NO:10), (S)ₓ (SEQ ID NO: 11), (C)ₓ (SEQ ID NO: 12), (Q)ₓ (SEQ ID NO: 13), (GD)ₓ (SEQ ID NO: 14), (GE)ₓ (SEQ ID NO: 15), (GQ)ₓ (SEQ ID NO:16), (GT)ₓ (SEQ ID NO:17), (L)ₓ (SEQ ID NO: 18), (LP)ₓ (SEQ ID NO: 19), (LPAC)_{X} (SEQ ID NO: 20), (LS)ₓ (SEQ ID NO: 21), (P)ₓ (SEQ ID NO: 22), (QAGR)_{X} (SEQ ID NO: 23), (RE)ₓ (SEQ ID NO: 24), (SP)ₓ (SEQ ID NO: 25), (VP)ₓ (SEQ ID NO: 26), (FP)ₓ (SEQ ID NO: 27), and/or (GK)ₓ (SEQ ID NO: 28) di-amino acid repeat, wherein x represents the number of repeat units of the antigen. In some embodiments, “x” is 5, 10, 15, 20, 25, 30, 35, 40, or more than 40 (e.g., 5, 10, 15, 20, 25, 30, 35, 40, or more than 40 repeat units). In some embodiments, a peptide antigen (e.g., a DPR peptide antigen) comprises between 2 and 150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150) amino acid repeats (e.g., different homopolymeric amino acid repeats, di-amino acid repeats, tri-amino acid repeats, tetra-amino acid repeats, penta-amino acid repeats, or any combination of the foregoing). In some embodiments, a peptide antigen (e.g., a DPR peptide antigen) comprises more than 50 amino acid repeats (e.g., different homopolymeric amino acid repeats, di-amino acid repeats, tri-amino acid repeats, tetra-amino acid repeats, penta-amino acid repeats, or any combination of the foregoing). In some embodiments, each DPR peptide antigen comprises between 10 di-amino acid repeats and 25 di-amino acid repeats. In some embodiments, a DPR peptide antigen comprises one of the following peptide antigens: (GA)₁₀(SEQ ID NO: 4), (GA)₁₅ (SEQ ID NO: 4), (GR)₂₅ (SEQ ID NO: 7), (GP)₁₀ (SEQ ID NO: 5), (PR)₁₀(SEQ ID NO: 6), or a combination thereof.

The number of peptide antigens of a RAN protein vaccine may vary (e.g., a RAN protein vaccine may be multivalent). In some embodiments, a RAN protein vaccine comprises peptide antigens to 2, 3, 4, 5, 6, 7, 8, 9, 10, 11, 12, or more than 12 different amino acid repeats (e.g., different homopolymeric amino acid repeats, di-amino acid repeats, tri-amino acid repeats, tetra-amino acid repeats, penta-amino acid repeats, or any combination of the foregoing). In some embodiments, a DPR peptide antigen comprises [(GA)₁₅ (SEQ ID NO: 4)+(GR)₂₅ (SEQ ID NO: 7)+(PR)₁₀ (SEQ ID NO: 6)] or [(GA)₁₅ (SEQ ID NO: 4)+(GR)₂₅ (SEQ ID NO: 7)+(PR)₁₀ (SEQ ID NO: 6)+(GP)₁₀ (SEQ ID NO: 5)].

Aspects of the disclosure relate to compositions (e.g., RAN protein vaccines, etc.) that comprise one or more (e.g., 1, 2, 3, 4, 5, 6, 7, 8, 9, 10, or more than 10) additional immunogens. An “immunogen” refers to any antigen that is capable of inducing humoral and/or cell-mediated immune response rather than immunological tolerance. Examples of immunogens include but are not limited to keyhole limpet hemocyanin (KLH), Blue Carrier Immunogenic protein (CCH), bovine serum albumin (BSA), ovalbumin (OVA), diphtheria toxin, measles virus fusion protein (MVF), hepatitis B virus surface antigen (HB-sAg), tetanus toxin (TT), pertussis toxin (PT), or a portion of any of the foregoing.

An immunogen may comprise a T helper cell epitope. A “T helper cell epitope” or “Th epitope” refers to T cell epitopes that are presented on the surface of an antigen-presenting cell, where they are bound to MHC class II molecules and are 13 to 17 amino acids in length, which are specifically recognized by T helper cells. In some embodiments, inclusion of a Th epitope in a vaccine results in a biased Th2 type regulatory T cell response in preference to Th1 pro-inflammatory T cell response in a vaccinated subject.

A peptide antigen and an additional immunogen (e.g., a B cell epitope and a T cell epitope) may be linked by any suitable modality. In some embodiments, the peptide antigen and the additional immunogen are covalently or non-covalently linked. In some embodiments, the peptide antigen and the additional immunogen are directly connected to one another. In some embodiments, a peptide antigen and an additional immunogen are linked via one or more (e.g., 1, 2, 3, 4, or more) linking molecules. In some embodiments, the conformational separation provided by one or more linking molecules permits more efficient interactions between the presented peptide antigen (e.g., DPR peptide antigen) and the appropriate Th cells and B cells, and thus enhances the immunogenicity of the peptide antigen (e.g., DPR peptide antigen) or cross-reactive functional immunological analogues thereof.

A linking molecule may provide a chemical linkage (e.g., linkage between one or more small molecules) or an amino acid linkage (e.g. linkage between amino acids, reactive groups of amino acids, hybridization, etc.), or a combination thereof. In some embodiments, a linking molecule comprises an amino acid linker. In some embodiments, a linking molecule is selected from the group consisting of an amino acid, Lys-, Gly-, Lys-Lys-Lys-, (a, F-N)Lys, and F-N-Lys-Lys-Lys-Lys (SEQ ID NO: 29).

In some embodiments, a RAN protein vaccine comprises one or more additional components, for example an N-terminal amide or signal peptide, or a C-terminal α-COOH or α-CONH₂.

A RAN protein vaccine or RAN protein immunogen may be formulated in a pharmaceutical composition. In some embodiments, the pharmaceutical composition comprises one or more pharmaceutically acceptable excipients. Pharmaceutical excipients are known, and are described for example by Remington, J. P. (1965). Remington's Pharmaceutical Sciences, Easton, Pa: Mack Pub. Co., 19th ed., 1995. In some embodiments, a pharmaceutical composition comprising a vaccine includes one or more adjuvants. An adjuvant is a pharmacological or immunological agent that modifies the effect of other agents. Adjuvants may be added to a vaccine to boost the immune response to produce more antibodies and longer-lasting immunity, thus minimizing the dose of antigen needed. In some embodiments, an adjuvant is a mineral salt of aluminum. In some embodiments an adjuvant is alhydrogel (Al(OH)₃) or adjuphos (AlPO₄). Aluminum-based adjuvants are known, for example as described in Shardlow et al. (2018) Allergy, Asthma & Clinical Immunology, 14:80. In some embodiments, a pharmaceutical composition further comprises CpG. In some embodiments, the CpG is a CpG oligodeoxynucleotide (ODN), for example as described by Weiner, et al., (1997) PNAS 94(20):10833-7. Adjuvants are discussed in greater detail in the section titled “Adjuvants,” below.

Vaccine compositions and immunogens, such as polypeptides and nucleic acids, that can be used in accordance with the present invention can be provided with a pharmaceutically-acceptable carrier or diluent. Compounds and compositions useful in the subject invention can be formulated according to known methods for preparing pharmaceutically useful compositions. Formulations are described in detail in a number of sources, which are well known and readily available to those skilled in the art. For example, Remington's Pharmaceutical Sciences, Easton Pa.: Mack Pub. Co., 19th ed., 1995, describes formulations which can be used in connection with the subject invention. In general, the compositions of the subject invention will be formulated such that an effective amount of an immunogen is combined with a suitable carrier in order to facilitate effective administration of the composition. The compositions used in the present methods can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the subject peptidomimetics include, but are not limited to, water, saline, oils including mineral oil, ethanol, dimethyl sulfoxide, gelatin, cyclodextrans, magnesium stearate, dextrose, cellulose, sugars, calcium carbonate, glycerol, alumina, starch, and equivalent carriers and diluents, or mixtures of any of these. Formulations of an immunogen of the invention can also comprise suspension agents, protectants, lubricants, buffers, preservatives, and stabilizers. To provide for the administration of such dosages for the desired therapeutic treatment, pharmaceutical compositions of the invention will advantageously comprise between about 0.1% and 45%, and especially, 1 and 15% by weight of the immunogen or immunogens based on the weight of the total composition including carrier or diluent.

The vaccine and immunogenic compositions of the subject invention can be prepared by procedures well known in the art. For example, the vaccine or immunogens are typically prepared as injectables, e.g., liquid solutions or suspensions. The vaccine or immunogens are administered in a manner that is compatible with dosage formulation, and in such amount as will be therapeutically effective and immunogenic in the recipient. The optimal dosages and administration patterns for a particular vaccine or immunogens formulation can be readily determined by a person skilled in the art.

In some embodiments, immunogens of the present invention can also be presented on a polysaccharide or other suitable carrier or scaffold. This structure of such a vaccine, without being bound by theory, is believed to induce greater immunogenicity and immune memory in the host, thereby facilitating an increase in immunogenic response due to the increased local concentration of antigen as encountered by immune system cells.

### Methods of Making Antigenic Peptides and Polypeptides of the Invention

The peptides of the present invention can be produced using any techniques available to those of ordinary skill in the art, such as chemical and biochemical synthesis. Examples of techniques for chemical synthesis of peptides are provided in Lee, Peptide and Protein Drug Delivery, New York, N.Y., Dekker (1990), in Ausubel, Current Protocols in Molecular Biology, John Wiley, 1987-1998, and in Sambrook et al. (1989).

In some embodiments, the peptide antigens of the present invention may be produced synthetically or naturally, for example as a recombinant protein vaccine. A “recombinant protein vaccine” is a vaccine whose active ingredient includes at least one protein antigen that is produced by recombinant expression. The vaccine antigens may be produced in bacteria, mammalian cells, baculovirus cells, and/or plant cells, or hybrids thereof, for example.

In some embodiments, the peptide antigens, or portions thereof, may be produced or manufactured totally synthetically, e.g., in cell-free translation systems or by chemical protein synthesis. The target antigens of the present invention may be obtained through any of the routes that are well known or available to those of ordinary skill in the art, including for example, recombinant production in vivo and chemical synthesis (e.g., SPPS). Recombinant production in vivo refers to the harvesting of protein from eukaryotic or prokaryotic cell cultures wherein the cells contain heterologous nucleotide coding sequences, typically within a plasmid under control of regulatory sequences, that code for one or more of the antigenic peptides or polypeptides of the present invention. Those of ordinary skill in the art will be capable of preparing such a plasmid using the genetic code to determine the nucleotide sequence necessary to encode the desired polypeptide sequence in combination with well-known regulatory sequences and commercially available plasmid vectors. Such systems are well known in the art, and standard recombinant DNA and molecular cloning techniques usable in connection with the present invention are known in the art and are described more fully, e.g., in Sambrook et al. (1989).

Preparation of peptide-based vaccines is generally well understood by those of ordinary skill in the art, and can be accomplished by a variety of available techniques, including, for example, those described in U.S. Pat. Nos. 4,608,251; 4,601,903; 4,599,231; 4,599,230; and 4,596,792; and generally as provided in Remington's Pharmaceutical Sciences, 16th Edition, A. Osol, (ed.), Mack Publishing Co., Easton, Pa. (1980), and Remington's Pharmaceutical Sciences, 19th Edition, A. R. Gennaro, (ed.), Mack Publishing Co., Easton, Pa. (1995).

The antigens and immunogenic compositions disclosed herein may be prepared by any method available to those of ordinary skill in the art. In some embodiments, the antigenic peptides and/or immunogens are presented with single or repeated occurrences of additional immunogenic components. Such additional components may be joined into the target antigens by way of collinear expression (i.e., fusion protein synthesis) or by chemical conjugation; such methods are well known in the art. In some embodiments, target antigens are presented on or in cells, organisms, and/or incomplete organisms (e.g., adeno-like viral particles) to provide enhanced immune response. In some embodiments, the target antigens may be synthesized in vivo as products of genetic manipulation of the host cells, i.e., as contained within a vector (e.g., recombinant viral vector), on a vector, or as a naked DNA or RNA vaccine. Techniques for preparing recombinant vaccines and delivering DNA/RNA vaccines are well known in the art and are discussed, for example, in U.S. Pat. Nos. 7,223,409 and 6,603,998.

### Adjuvants

In some embodiments, the antigen peptides and immunogens of the present invention are sufficiently immunogenic to induce an immune response when presented in a vaccine. In some embodiments, the immune response can be increased if the immunogenic composition (e.g., vaccine) further includes an adjuvant substance. The term “adjuvant” refers to a substance or a composition of matter that is: (1) not in itself capable of mounting a specific immune response against the immunogen of the vaccine, but which is (2) nevertheless capable of enhancing the immune response against the immunogen. In some embodiments, combined vaccination with the peptide antigen and/or immunogen and an adjuvant induces an immune response against the peptide antigen and/or immunogen that is stronger than that induced by the peptide antigen and/or immunogen alone (e.g., without the adjuvant).

Various methods of achieving an adjuvant effect for the vaccine are known. General principles and methods suitable for use in accordance with the invention are detailed in “The Theory and Practical Application of Adjuvants,” Duncan E. S. Stewart-Tull (Eds.), John Wiley & Sons Ltd, Malden, Mass., USA (1995); and in “Vaccines: New Generation Immunological Adjuvants,” Gregoriadis et al., (Eds.), Plenum Press, New York, N.Y., USA (1995).

In some embodiments, an adjuvant is one that correlates to and/or causes a “stimulation of the immune system.” A stimulation of the immune system means that a substance or composition of matter exhibits a general, non-specific immunostimulatory effect. A number of adjuvants and putative adjuvants (such as certain cytokines) share the ability to stimulate the immune system. In some embodiments, the result of using an immunostimulating agent is an increased “alertness” of the immune system (e.g., simultaneous or subsequent immunization with a peptide antigen and/or immunogen can induce a significantly more effective immune response compared to isolated use of the peptide antigen and/or immunogen). Complete Freund's adjuvant (“CFA”) is an adjuvant that in some embodiments may be used in association with vaccines of the present invention. Other adjuvants that may be used include those that would drive levels of increased protective immunity through either the cellular or the humoral system.

Non-limiting examples of adjuvants for use in the present invention include: one or more immune targeting adjuvants; immunomodulating adjuvants such as a toxin, a cytokine, and a mycobacterial derivative; incomplete Freund's adjuvant; aluminum phosphate; aluminum hydroxide; alum; Stimulon® QS-21 (Aquila Biopharmaceuticals, Inc., Framingham, Mass., USA); MPL® (3, 0, deacylated monophosphoryl lipid A; Corixa Corp., Hamilton, Mont., USA); interleukin-12 (Genetics Institute, Cambridge, Mass., USA); an oil formulation; a polymer; a micelle forming adjuvant; a saponin; an immunostimulating complex matrix (ISCOM matrix); a particle; DDA; DNA adjuvants; an encapsulating adjuvant; or any of the adjuvants as described in U.S. Pat. Nos. 7,357,936; 7,090,853; 6,793,928; 6,780,421; 6,759,241; 6,713,068; 6,572,866; 6,534,065; 6,451,325; 6,440,423; 6,306,404; 6,060,068; 6,033,673; 5,800,810; 5,795,582; 5,785,975; 5,679,356; 5,503,841; and 5,182,109. Adjuvants may also include sterile liquids such as water, saline, petroleum oil, vegetable oil, soybean oil, peanut oil, and/or mineral oil, or a combination thereof. In some embodiments, the adjuvant is CpG3.

In some embodiments, an adjuvant stimulates T-cells (e.g., detoxified heat-labile E. coli enterotoxin adjuvant). In some embodiments, aluminum-based adjuvants (e.g., aluminum hydroxide and aluminum phosphate) are used. Aluminum-based adjuvants (e.g., aluminum hydroxide or aluminum phosphate) are commonly used as about 0.05 to 0.1% solution in buffered saline, admixture with synthetic polymers of sugars (e.g., Carbopol® Noveon, Inc. Cleveland, Ohio, USA) used as 0.25% solution, aggregation of the protein in the vaccine by heat treatment with temperatures ranging from about 70° C. to about 101° C. for about 30 sec to about 2 min periods, respectively, and also aggregation by means of cross-linking agents is also possible. Aggregation by reactivation with pepsin-treated antibodies (including, for example, Fab fragments) to albumin, mixture with bacterial cells such as C. parvum, endotoxins, or lipopolysaccharide components of Gram-negative bacteria, emulsion in physiologically acceptable oil vehicles such as mannide mono-oleate (Aracel-A®, Sigma Chemical Co.), or emulsion such as with 20% solution of a perfluorochemical blood substitute (e.g., Fluosol-DA®), or any combination thereof, may also be employed. Admixture with oils such as squalene and IFA may also be employed.

The antigenic compositions of the invention may be conjugated to or expressed on one or more carriers. U.S. Pat. Appl. Publ. No. 2004/0223976 discusses exemplary methods for conjugation. For conjugation, a carrier may in some embodiments be a fungus, a bacterium, a virus or a virus-like particle or a portion thereof, a protein or protein complex (e.g., complete or incomplete capsid particle), a polysaccharide or polysaccharide complex, a polynucleotide or polynucleotide complex (double helix, triple helix, hairpin loop, etc.), an organic or inorganic polymer, a microbead or microsphere, a nanoparticle or nanosphere, a ballistic particle, etc., and/or any combination thereof. As used herein, a “carrier protein” means an immunogenic or nonimmunogenic protein to which the peptide and polypeptide sequences, polysaccharides, and/or other antigens are conjugated. Various immunogenic carrier proteins are known in the art and may be used in conjugate vaccines. Carrier proteins include, but are not limited to, the outer membrane protein complex (OMPC) of Neisseria meningitidis, tetanus toxoid protein, hepatitis B virus proteins (including, for example, the surface antigen protein [HBsAg] and the core antigen protein [HB CoreAg]), keyhole limpet hemocyanin (KLH), rotavirus capsid proteins, and the L1 protein of a bovine papillomavirus VLP or human papillomavirus VLP (e.g., VLPs of HPV type 6, 11 or 16, etc, or pharmaceutically acceptable salts thereof), or any combination of the foregoing.

The peptide antigens and/or immunogens of the present disclosure may in some embodiments be expressed on the surface of microbes, e.g., adenovirus, adeno-like virus, or even incomplete viral particles such as a portion of the foregoing or other virus particles.

### Adjuvanted Antigenic Peptide Compositions

Although the peptide antigens, immunogens, and compositions thereof of the invention may be formulated alone, in other embodiments they may also be formulated as polysaccharide vaccines, protein-polysaccharide vaccines, conjugated vaccines, or together as fusion proteins (e.g., with HA or NA sequences, or both). Conjugates may take the form of a microbial or viral antigen. A “microbial or viral antigen,” as used herein, refers to an antigen or epitopic sequence of a microorganism or a virus, and includes, but is not limited to, infectious or pathogenic virus, bacteria, yeast, fungi, parasites, amoebae, or any combination thereof. Such antigens may include the intact virus or microorganism itself, as well as natural isolates, fragments, capsids, cell fractions, membrane components, lysates, or derivatives thereof, in addition to synthetic or recombinant compounds which are identical, or similar (i.e., substantially homologous), to a native (i.e., in vivo or in situ) antigen obtained from such a microorganism or virus. In all such cases, an antigen preferably will illicit an immune response in at least a first animal when presented to the immune system of the animal host.

In some embodiments of the invention involving polyvalent or polymicrobial immunogens and vaccines, the immunogen may comprise at least two distinct antigens, at least one of which is specific for a particular microbe or virus. In the case of intraspecies polyvalent immunogens, such compositions will preferably comprise at least two different epitopes or antigens that elicit an immune response against two or more species of a given organism. Alternatively, in the case of interspecies polyvalent immunogens, such compositions will preferably comprise at least two different epitopes or antigens that elicit an immune response against at least one species from one organism, and that also elicit an immune response against at least one species from a second different organism.

A compound is similar to a natural microorganism antigen if it induces an immune response (humoral and/or cellular) to a natural microorganism antigen. Such antigens are used routinely in the art and are well known to the ordinary-skilled artisan, and may include a fungus, bacterium, a virus, or virus-like particle, or a portion thereof, or a combination thereof. Generally, if vaccination with the conjugate reduces the level of infection or the severity of the resulting disease, then the peptide and conjugate is considered useful in the preparation of the vaccine. Moreover, any method available to those of ordinary skill in the art may be used to conjugate the immunogens and vaccines of the present invention with non-peptide compounds such as polysaccharides, lipids, lipopeptides, and the like.

Proteins may contain one or more T-cell epitopes that make them T-cell-dependent antigens capable of eliciting T-cell help when used as a vaccine antigen. Antigenic peptide and immunostimulatory amino acid sequences derived from diphtheria and tetanus toxoids, or any other T-cell epitope from any source, or portions or combinations of any of the foregoing and others, can also be used in the formulation of the immunogens and vaccines according to the invention.

The antigenic compositions of the present invention can be conjugated to carriers using any conjugation method in the art. For example, the conjugation can be achieved using sulfosuccinimidyl 4-(N-maleimidomethyl)cyclohexane-1-carboxylate (sSMCC), N-[ε-maleimidocaproyloxy]sulfosuccinimide ester (sEMCS), N-maleimidobenzoyl-N-hydroxysuccinimide ester (MBS), glutaraldehyde, 1-ethyl-3-(3-dimethylaminopropyl)carbodiimide (EDCI), Bis-diazobenzidine (BDB), or N-acetyl homocysteine thiolactone (NAHT), or a combination thereof.

In some embodiments, the immunogenic composition of the invention comprises microbial polysaccharides. Polysaccharides are of native size or alternatively may be sized, for instance by microfluidization, ultrasonic irradiation, or by chemical treatment. The invention also covers oligosaccharides derived from the type 5 and type 8 polysaccharides from S. aureus. In some embodiments, the immunogenic composition of the invention further comprises the polysaccharide PIA (or PNAG). PIA (or PNAG) may be of different sizes varying from over 400 kDa to between 75 and 40 kDa to between 10 and 75 kDa to oligosaccharides composed of up to 30 repeat units of (1→6)-β-D-glucosamine substituted with N-acetyl and O-succinyl constituents). The polypeptides may be linked to the carrier polysaccharides(s) by any known method (for example, by U.S. Pat. Appl. Publ. No. US2005/0169941; U.S. Pat. Nos. 4,372,945, 4,474,757, and 4,356,170).

In certain embodiments of the invention, conjugation reactions to couple the peptide to the carrier can involve introducing and/or using intrinsic nucleophilic groups on one reactant and introducing and/or using intrinsic electrophilic groups in the other reactant. For example, a nucleophilic thiol group can be introduced to the carrier protein (preferably OMPC) and then electrophilic groups (preferably alkyl halides or maleimide) can be added to the peptide. The resulting conjugate has thiol ether bonds linking the peptide and carrier. Direct reaction of the peptide electrophilic group (maleimide or alkyl halide) and intrinsic nucleophilic groups (preferably primary amines or thiols) of the carrier protein can lead to secondary amine linkages or thioether bonds. Alternative schemes involve adding a maleimide group or alkyl halide to the carrier, and introducing a terminal cysteine to the peptide and/or using intrinsic peptide thiols again can result in thiol ether linkages when desired.

### Exosomes

Aspects of the disclosure include the use of exosomes to identify certain peptide antigens and/or immunogens that are specific to the subject being administered the RAN protein vaccine or composition of the present disclosure. In some embodiments, subject-specific peptide antigens and/or immunogens of a RAN protein vaccine are identified in an exosome of the subject. When peptide antigens (e.g., for a vaccine) are identified in an exosome of the subject, such antigens are said to be representative of exosome antigens of the subject.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome and exhibit membrane proteins from the parental cell on the surface.

Exosomes exhibit different composition and function depending on the cell type from which they are derived. There are no “exosome-specific” proteins; however, several proteins identified in these vesicles are associated with endosomes and lysosomes reflecting their origin. Most exosomes are enriched in MHC I and II (major histocompatibility complex I and II; important for antigen presentation to immunocompetent cells such as T-lymphocytes), tetraspanins, several heat shock proteins, cytoskeletal components such as actins and tubulins, proteins involved in intracellular membrane fusion, signal transduction proteins, and cytosolic enzymes.

Exosomes are produced by many cells including epithelial cells, B and T lymphocytes, mast cells (MC), and dendritic cells (DC). In humans, exosomes have been found in blood, plasma, urine, bronchoalveolar lavage fluid, intestinal epithelial cells, and tumor tissues.

All functions of exosomes have not been elucidated, but data strongly indicates they mediate communication between cells. This communication could take place in different ways. First, exosomes could bind to cell surface receptor in a similar way as cell to cell interaction. Second, exosomes could attach to the cell membrane and give the cells new receptors and properties. Thus, exosomes can also fuse with target cells and exchange membrane proteins and cytosol between two cell types.

A number of methods of isolating exosomes from a biological sample have been described in the art. For example, the following methods can be used: differential centrifugation, low speed centrifugation, anion exchange and/or gel permeation chromatography, sucrose density gradients or organelle electrophoresis, magnetic activated cell sorting (MACS), nanomembrane ultrafiltration concentration, Percoll gradient isolation, and/or using microfluidic devices. Exemplary methods are described in US Patent Publication No. 2014/0212871, for example.

### Nanoparticle Formulations

In some embodiments, a RAN protein vaccine is formulated in a nanoparticle. In some embodiments, a RAN protein vaccine is formulated in a lipid nanoparticle. In some embodiments, a RAN protein vaccine is formulated in a lipid-polycation complex, referred to as a cationic lipid nanoparticle. As a non-limiting example, the polycation may include a cationic peptide or a polypeptide such as, but not limited to, polylysine, polyornithine, and/or polyarginine. In some embodiments, a RAN protein vaccine is formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components, and biophysical parameters such as size. In one example by Semple, et al., (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (see, e.g., Basha, et al., Mol Ther. 2011 19:2186-2200).

In some embodiments, lipid nanoparticle formulations may comprise 35 to 45% cationic lipid, 40% to 50% cationic lipid, 50% to 60% cationic lipid, and/or 55% to 65% cationic lipid.

In some embodiments, a RAN protein vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids, and amino alcohol lipids. In some embodiments, the lipid may be a cationic lipid such as, but not limited to, DLin-DMA, DLin-D-DMA, DLin-MC3-DMA, DLin-KC2-DMA, DODMA, and amino alcohol lipids.

The amino alcohol cationic lipid may be the lipids described in and/or made by the methods described in U.S. Patent Publication No. US20130150625, for example. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z, 12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol, and a molecule capable of reducing particle aggregation, for example a PEG or PEG-modified lipid.

mRNA RAN Protein Vaccines

Some aspects of the present invention contemplate RNA (e.g., mRNA) vaccines comprising the antigen peptides and/or immunogens as described herein. Without wishing to bound by any particular theory, RNA (e.g., mRNA) vaccines may have superior properties in that they produce much larger antibody titers and produce responses earlier than commercially available anti-viral therapeutic treatments. It is believed that the RNA (e.g., mRNA) vaccines, as mRNA polynucleotides, are better designed to produce the appropriate protein conformation upon translation as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

In some embodiments, a RAN protein vaccine of the present invention is an mRNA RAN protein vaccine. In some embodiments, the mRNA RAN protein vaccine comprises at least one RNA polynucleotide having an open reading frame encoding at least one peptide antigen or at least one immunogen as described herein. In some embodiments, the mRNA RAN protein vaccine is formulated in a cationic lipid nanoparticle.

In some embodiments, an mRNA RAN protein vaccine is combined with a flagellin adjuvant (e.g., one or more peptide antigen- or immunogen-encoding mRNAs is combined with an mRNA encoding flagellin). RNA (e.g., mRNA) vaccines combined with the flagellin adjuvant (e.g., mRNA-encoded flagellin adjuvant) have superior properties in that they may produce much larger antibody titers and produce responses earlier than commercially available vaccine formulations. In some embodiments, the mRNA RAN protein vaccine includes at least one RNA (e.g., mRNA) polynucleotide having an open reading frame encoding at least one antigenic peptide or an immunogenic fragment thereof (e.g., an immunogenic fragment capable of inducing an immune response to the antigenic peptide) and at least one RNA (e.g., mRNA polynucleotide) having an open reading frame encoding a flagellin adjuvant.

### Methods of Detecting Anti-RAN Antibodies in a Biological Sample

Aspects of the disclosure relate to detecting and/or measuring levels of anti-RAN antibodies present in one or more biological samples obtained from a subject. In some embodiments, the one or more anti-RAN protein antibodies is one or more of an anti-poly(CP), anti-poly(GA), anti-poly(GP), anti-poly(PR), anti-poly(GR), anti-poly(PA), anti-poly(A), anti-poly(G), anti-poly(S), anti-poly(C), anti-poly(Q), anti-poly(GD), anti-poly(GE), anti-poly(GQ), anti-poly(GT), anti-poly(L), anti-poly(LP), anti-poly(LPAC (SEQ ID NO: 1)), anti-poly(LS), anti-poly(P), anti-poly(QAGR (SEQ ID NO: 2)), anti-poly(RE), anti-poly(SP), anti-poly(VP), anti-poly(FP), and/or anti-poly(GK) antibody, or any combination thereof.

In some embodiments, the presence of one or more anti-RAN antibodies is detected and/or measured in a biological sample obtained from a subject. Methods of detecting antibodies in biological samples (e.g., blood, serum, and/or CSF samples, etc.) are known in the art, and include, for example, Enzyme-linked Immunosorbent assay (ELISA) (e.g., RCA-based ELISA, rtPCR-based ELISA, etc.), ElectroChemiLuminescence Immuno assay (Meso Scale Discovery, MSD), digital ELISA technology (Single molecule array, SIMOA), Western blot analysis, immunohistochemistry, binding assay, immunoblot, and/or Dot blot assay. In some embodiments, detection of one or more RAN proteins is performed by immunoblot (e.g., dot blot, 2-D gel electrophoresis, Western Blot, etc.), immunohistochemistry (IHC), ELISA (e.g., RCA-based ELISA or rtPCR-based ELISA), label free immunoassays such as surface plasmon resonance bio layer interferometry, immunoquantitative PCR, mass spectrometry such as GC-MS, LC-MS, MALDI-TOF-MS, bead-based immunoassays, immunoprecipitation, immunostaining, or immunoelectrophoresis.

Certain methods of detection (e.g., Western blots) employ the use of a detection agent or probe to identify the presence of a protein (e.g., an antibody, for example an anti-RAN antibody) or peptide. In some embodiments, the detection agent is an antigen. In some embodiments, the antigen targets one or more RAN proteins, optionally wherein the one or more RAN proteins is one or more of: poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), and/or poly(GK).

In some embodiments, the antigen comprises one or more di-peptide repeat (DPR) peptide antigens. In some embodiments, each DPR peptide antigen comprises between 2 di-amino acid repeats and 150 di-amino acid repeats. In some embodiments, each DPR peptide antigen comprises between 10 di-amino acid repeats and 25 di-amino acid repeats. In some embodiments, each DPR peptide antigen comprises between 25 di-amino acid repeats and 50 di-amino acid repeats. In some embodiments, each DPR peptide antigen comprises between 50 di-amino acid repeats and 100 di-amino acid repeats. In some embodiments, each DPR peptide antigen comprises between 100 di-amino acid repeats and 150 di-amino acid repeats. In some embodiments, each di-peptide repeat antigen comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 di-amino acid repeats.

In some embodiments, each of the one or more DPR peptide antigens comprises a (GA)ₓ (SEQ ID NO: 4), (GP)ₓ (SEQ ID NO: 5), (GR)ₓ (SEQ ID NO: 7), (PA)ₓ (SEQ ID NO: 8), or (PR)ₓ (SEQ ID NO: 6) di-amino acid repeat, wherein x represents the number of repeat units of the antigen. In some embodiments, each of the one or more DPR peptide antigens comprises a (CP)ₓ (SEQ ID NO:3), (A)ₓ (SEQ ID NO:9), (G)ₓ (SEQ ID NO:10), (S)ₓ (SEQ ID NO: 11), (C)ₓ (SEQ ID NO: 12), (Q)ₓ (SEQ ID NO: 13), (GD)ₓ (SEQ ID NO: 14), (GE)ₓ (SEQ ID NO: 15), (GQ)ₓ (SEQ ID NO:16), (GT)ₓ (SEQ ID NO:17), (L)ₓ (SEQ ID NO: 18), (LP)ₓ (SEQ ID NO: 19), (LPAC)_{X} (SEQ ID NO: 20), (LS)ₓ (SEQ ID NO: 21), (P)ₓ (SEQ ID NO: 22), (QAGR)_{X}(SEQ ID NO: 23), (RE)ₓ (SEQ ID NO: 24), (SP)ₓ (SEQ ID NO: 25), (VP)ₓ (SEQ ID NO: 26), (FP)ₓ (SEQ ID NO: 27), and/or (GK)ₓ (SEQ ID NO: 28) di-amino acid repeat, wherein x represents the number of repeat units of the antigen. In some embodiments, x is 5, 10, 15, 20, 25, 30, 35, or 40, or more than 40 (e.g., 5, 10, 15, 20, 25, 30, 35, 40, or more than 40 repeat units). In some embodiments, the one or more peptide antigens comprises a B-cell epitope.

In some embodiments, a DPR peptide antigen comprises (GA)₁₀(SEQ ID NO: 4), (GA)₁₅ (SEQ ID NO: 4), (GR)₂₅ (SEQ ID NO: 7), (GP)₁₀ (SEQ ID NO: 5), (PR)₁₀ (SEQ ID NO: 6), or a combination thereof. In some embodiments, a DPR peptide antigen comprises [(GA)₁₅ (SEQ ID NO: 4)+(GR)₂₅ (SEQ ID NO: 7)+(PR)₁₀ (SEQ ID NO: 6)]. In some embodiments, a DPR peptide antigen comprises [(GA)₁₅ (SEQ ID NO: 4)+(GR)₂₅ (SEQ ID NO: 7)+(PR)₁₀ (SEQ ID NO: 6)+(GP)₁₀ (SEQ ID NO: 5)].

### Methods of Eliciting Antibody Production, Monitoring Disease Progression, and Evaluating Treatment Efficacy

Aspects of the disclosure relate to methods for eliciting (e.g., promoting or enhancing) expression or production of anti-RAN protein antibodies in a subject. In some embodiments, the disclosure provides a method for increasing or enhancing expression or production of anti-RAN protein antibodies in a subject, the method comprising administering to the subject a RAN protein vaccine and/or RAN protein immunogen as described herein. In some embodiments, administration of a RAN protein vaccine and/or RAN protein immunogen results in an elevated level of anti-RAN protein antibodies in the subject. In some embodiments, administration of a RAN protein vaccine and/or RAN protein immunogen results in an unchanged or a decreased level of anti-RAN protein antibodies in the subject.

Further aspects of the disclosure relate to monitoring the level of one or more anti-RAN antibodies present in a biological sample obtained from a subject having or suspected of having a RAN-protein associated disease or disorder longitudinally over a course of treatment (e.g., over a specified period of time), thereby providing an assessment of therapeutic efficacy of certain treatments (e.g., vaccines) for diseases and disorders associated with RAN protein expression, translation, and/or accumulation. In some embodiments a subject has previously been administered a RAN protein vaccine and/or RAN protein immunogen as described herein. In some embodiments, the disease or disorder associated with RAN proteins is selected from the group consisting of: amyotrophic lateral sclerosis (ALS), frontotemporal dementia (FTD), myotonic dystrophy type 1 (DM1), myotonic dystrophy type 2 (DM2), spinocerebellar ataxia types 1, 2, 3, 6, 7, 8, 10, 12, 17, 31, and 36, spinal bulbar muscular atrophy, dentatorubral-pallidoluysian atrophy (DRPLA), Huntington's disease (HD), Alzheimer's disease (AD), Fragile X Tremor Ataxia Syndrome (FXTAS), Fuch's endothelial corneal dystrophy (FECD), Huntington's disease-like 2 syndrome (HDL2), Fragile X syndrome (FXS), disorders related to 7pl 1 0.2 folate-sensitive fragile site FRA7A, disorders related to folate-sensitive fragile site 2ql 1 FRA2A, and Fragile XE syndrome (FRAXE).

In some embodiments, one or more anti-RAN protein antibodies are detected in a biological sample obtained from a subject having or suspected of having a RAN-protein associated disease or disorder according to the methods described herein.

In some embodiments, a first biological sample is obtained from the subject prior to the administration of a RAN protein vaccine and/or RAN protein immunogen as described herein (e.g., prior to a therapeutic regimen), and a second biological sample is obtained from the subject after administration of a RAN protein vaccine and/or RAN protein immunogen as described herein. In some embodiments, the first biological sample is a control sample (e.g., a control blood, serum, or CSF sample). In some embodiments, a control sample is a prior sample screened in the same subject having or suspected of having a RAN-protein associated disease or disorder (e.g., a sample taken from the same subject 1 hour earlier than the second sample, 1 day earlier, 2 days earlier, 3 days earlier, 4 days earlier, 5 days earlier, 6 days earlier, 1 week earlier, 2 weeks earlier, 3 weeks earlier, 1 month earlier, 2 months earlier, 3 months earlier, 6 months earlier, 1 year earlier, 2 years earlier, 3 years earlier, 4 years earlier, 5 years earlier, 10 years earlier, 20 years earlier, etc.). In some embodiments, a control sample is a later sample screened in the same subject having or suspected of having a RAN-protein associated disease or disorder (e.g., a sample taken from the same subject 1 hour later than the first sample, 1 day later, 2 days later, 3 days later, 4 days later, 5 days later, 6 days later, 1 week later, 2 weeks later, 3 weeks later, 1 month later, 2 months later, 3 months later, 6 months later, 1 year later, 2 years later, 3 years later, 4 years later, 5 years later, 10 years later, 20 years later, etc.). In some embodiments, the control sample is taken from a subject who has not been diagnosed with, and has no visible, noticeable, or otherwise phenotypic symptoms of, a RAN-protein associated disease or disorder (e.g., a healthy control subject). In some embodiments, a control sample is a sample taken from a different subject having a RAN-protein associated disease or disorder. In some embodiments, a control sample is a sample taken from a control subject that is matched (e.g., age-matched, gender-matched, etc.) to the subject having or suspected of having a RAN-protein associated disease or disorder.

The time between which a first biological sample and a second biological sample are obtained may vary. In some embodiments, a first biological sample is obtained between 1 week and 1 minute prior to administration of a therapeutic agent (e.g., the first administration of a therapeutic agent, such as a RAN protein vaccine). In some embodiments, a first biological sample is obtained between 1 day (e.g., 24 hours) and 1 minute prior to administration of a therapeutic agent (e.g., the first administration of a therapeutic agent). In some embodiments, a second biological sample is obtained from the subject between 1 minute and six months after administration of a therapeutic agent (e.g., the first administration of a therapeutic agent). In some embodiments, a second biological sample is obtained from the subject between 1 day and 1 week after administration of a therapeutic agent (e.g., the first administration of a therapeutic agent). In some embodiments, a second biological sample is obtained from the subject between 1 day and 1 week after administration of a therapeutic agent (e.g., the most recent or last administration of a therapeutic agent).

In some embodiments, a second biological sample may be collected about 1 hour, 5 hours, 10 hours, 24 hours (e.g., 1 day), 48 hours (e.g., 2 days), 120 hours (e.g., 5 days), 30 days, 45 days, or six months after administration of the therapeutic agent. In some embodiments, several biological samples (e.g., 2, 3, 4, 5, 6, 7, 8, 9, 10, or more than 10 biological samples) are obtained from the subject, for example over a specified timeframe (e.g., during a therapeutic regimen), and one or more anti-RAN protein antibodies are detected according to the methods described herein.

As used herein, “elevated” means that the level of one or more anti-RAN antibodies present in a biological sample (e.g., a serum sample) is above a control level, such as a pre-determined threshold or a level of one or more anti-RAN protein antibodies in a control sample. Controls and control levels include anti-RAN protein antibody levels obtained (e.g., detected) from a subject that does not have or is not suspected of having a disease or disorder associated with RAN protein expression, translation, and/or accumulation (e.g., ALS/FTD or SCA36, and/or a subject having 30 or less repeats of a GGGGCC expansion or a TGGGCC expansion). In some embodiments, a control or control level includes anti-RAN protein antibody levels prior to administration of a therapeutic agent (e.g., a RAN protein vaccine and/or RAN protein immunogen). An elevated level includes a level that is, for example, 1%, 5%, 10%, 20%, 30%, 40%, 50%, 60%, 70%, 80%, 90%, 100%, 150%, 200%, 300%, 400%, 500%, or more than 500% above a control level. An elevated level also includes increasing a phenomenon from a zero state (e.g., no or undetectable anti-RAN protein antibody expression or level) to a non-zero state (e.g., some or detectable level of anti-RAN protein antibody expression or presence). In some embodiments, an increase (e.g., increase in the level of one or more anti-RAN protein antibody levels in the sample relative to a control or a prior sample) can be indicative of the therapeutic efficacy of a therapeutic agent (e.g., therapeutic efficacy in the subject from which the sample was obtained). In some embodiments, measuring an elevated level of one or more anti-RAN protein antibodies in a subject after administration of a therapeutic agent for treatment of a disease or disorder associated with RAN protein expression, translation, and/or accumulation (e.g., ALS/FTD) (e.g., relative to the level of anti-RAN protein antibodies measured in the subject prior to the administration) is indicative of the therapeutic agent effectively treating the subject for the disease or disorder associated with RAN protein expression, translation, and/or accumulation (e.g., ALS/FTD).

As used herein, “unchanged or decreased” means that the level of one or more anti-RAN protein antibody is at or below a control level, such as a pre-determined threshold or a level of one or more anti-RAN protein antibodies in a control sample. Controls and control levels include RAN protein levels obtained (e.g., detected) from a subject that does not have or is not suspected of having a disease or disorder associated with RAN protein expression, translation, and/or accumulation (e.g., ALS/FTD, and/or a subject having 30 or less repeats of a GGGGCC expansion). In some embodiments, a control or control level includes anti-RAN protein antibody levels prior to administration of a therapeutic agent (e.g., a RAN protein vaccine and/or RAN protein immunogen). An unchanged level is a level that is the same as a control level. A decreased level includes a level that is, for example, 1%, 5%, 10%, 20%, 30%, 40%, 50%, 60%, 70%, 80%, 90%, 100%, 150%, 200%, 300%, 400%, 500%, or more than 500% below a control level. A decreased level also includes decreasing a phenomenon from a non-zero state (e.g., some or detectable anti-RAN protein antibody expression or presence) to a zero state (e.g., no or undetectable anti-RAN protein antibody expression or presence). In some embodiments, a lack of change or a decrease (e.g., lack of change or decrease in the level of one or more anti-RAN protein antibody levels in the sample relative to a control or a prior sample) can be indicative of a lack of therapeutic efficacy of a therapeutic agent (e.g., a lack of therapeutic efficacy in the subject from which the sample was obtained). In some embodiments, measuring a lack of change or a decreased level of one or more anti-RAN protein antibodies in a subject after administration of a therapeutic agent for the treatment of a disease or disorder associated with RAN protein expression, translation, and/or accumulation (e.g., ALS/FTD) (e.g., relative to the level of anti-RAN protein antibodies measured in the subject prior to the administration) is indicative of the therapeutic agent not effectively treating the subject for the disease or disorder associated with RAN protein expression, translation, and/or accumulation (e.g., ALS/FTD).

As used herein, a “change” in one or more anti-RAN protein antibody levels in the subject occurs if the amount of anti-RAN protein antibodies detected in the first biological sample is different than the amount of anti-RAN protein antibodies detected in the second biological sample. The amount of anti-RAN protein antibodies detected in the first biological sample is considered “different” than the amount of anti-RAN protein antibodies detected in the second biological sample when either an elevated or a decreased level of one or more anti-RAN protein antibodies is observed in the second biological sample relative to the first biological sample.

In some embodiments, if the level (e.g., amount) of anti-RAN protein antibodies detected in the post-treatment sample is increased compared to the pre-treatment level (e.g., amount) of anti-RAN protein antibodies, the therapeutic regimen is successful. In some embodiments, if the level (e.g., amount) of anti-RAN protein antibodies detected in the post-treatment sample is decreased compared to the pre-treatment level (e.g., amount) of anti-RAN protein antibodies, the therapeutic regimen is not successful. In some embodiments, the level of anti-RAN protein antibodies in biological samples (e.g., 1, 2, 3, 4, 5, 6, 7, 8, 9, 10, or more than 10 samples) obtained from a subject are continuously monitored during a therapeutic regimen (e.g., measured on 1, 2, 3, 4, 5, 6, 7, 8, 9, 10, or more than 10separate occasions).

In some embodiments, methods described by the disclosure comprise a step of administering (or continuing to administer) a therapeutic agent (e.g., an agent for treatment of a disease associated with RAN protein expression, translation, and/or accumulation, such as ALS/FTD, for example a RAN protein vaccine and/or RAN protein immunogen) to the subject if the level of anti-RAN protein antibodies detected in the biological sample (e.g., the second biological sample) is elevated compared to a level of RAN proteins detected in a control sample (e.g., the first biological sample). In some embodiments, methods described by the disclosure comprise a step of stopping the administration of a therapeutic agent (e.g., an agent for treatment of a disease or disorder associated with RAN protein expression, translation, and/or accumulation, such as ALS/FTD) to the subject if the level of anti-RAN protein antibodies detected in the biological sample (e.g., the second biological sample) is decreased compared to a level of RAN proteins detected in a control sample (e.g., the first biological sample).

Methods of Preventing or Treating a Disease Associated with RAN Proteins

In some aspects, the disclosure provides a method for preventing or treating a disease associated with RAN protein translation in a subject in need thereof, the method comprising administering to the subject a therapeutically effective amount of a RAN protein vaccine, wherein administration of the RAN protein vaccine to the subject elicits production of one or more anti-RAN protein antibodies in the subject. In some embodiments, the one or more anti-RAN protein antibodies is one or more of an anti-poly(CP), anti-poly(GA), anti-poly(GP), anti-poly(PR), anti-poly(GR), anti-poly(PA), anti-poly(A), anti-poly(G), anti-poly(S), anti-poly(C), anti-poly(Q), anti-poly(GD), anti-poly(GE), anti-poly(GQ), anti-poly(GT), anti-poly(L), anti-poly(LP), anti-poly(LPAC (SEQ ID NO: 1)), anti-poly(LS), anti-poly(P), anti-poly(QAGR (SEQ ID NO: 2)), anti-poly(RE), anti-poly(SP), anti-poly(VP), anti-poly(FP), and/or anti-poly(GK) antibody, or any combination thereof.

In some embodiments, a disease associated with RAN proteins is amyotrophic lateral sclerosis (ALS), Huntington's disease (HD), Alzheimer's disease (AD), Fragile X Syndrome (FRAXA), Spinal Bulbar Muscular Atrophy (SBMA), Dentatorubropallidoluysian Atrophy (DRPLA), Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17), amyotrophic lateral sclerosis (ALS), Spinocerebellar ataxia type 36 (SCA36), Spinocerebellar ataxia type 29 (SCA29), Spinocerebellar ataxia type 10 (SCA10), myotonic dystrophy type 1 (DM1), myotonic dystrophy type 2 (DM2), or Fuch's Corneal Dystrophy (e.g., CTG181).

As used herein, “treat” or “treatment” refers to (a) preventing or delaying the onset of a disease or disorder associated with RAN protein expression, translation, and/or accumulation; (b) reducing the severity of a disease or disorder associated with RAN protein expression, translation, and/or accumulation; (c) reducing or preventing development of symptoms characteristic of a disease or disorder associated with RAN protein expression, translation, and/or accumulation; (d) preventing worsening of symptoms characteristic of a disease or disorder associated with RAN protein expression, translation, and/or accumulation; and/or (e) reducing or preventing recurrence of symptoms in subjects that were previously symptomatic for a disease or disorder associated with RAN protein expression, translation, and/or accumulation.

For example, in the context of ALS/FTD or SCA36, “treat” or “treatment” refers to (a) preventing or delaying the onset of ALS and/or FTD, or SCA36; (b) reducing the severity of ALS and/or FTD, or SCA36; (c) reducing or preventing development of symptoms characteristic of ALS and/or FTD, or SCA36; (d) preventing worsening of symptoms characteristic of ALS and/or FTD, or SCA36; and/or (e) reducing or preventing recurrence of ALS and/or FTD, or SCA36 symptoms in subjects that were previously symptomatic for ALS and/or FTD, or SCA36.

In some embodiments, a subject is administered one or more additional therapeutic agents, in conjunction with (e.g., before, at the same time as, or after) one or more therapeutic agents of the present disclosure (e.g. a RAN protein vaccine and/or RAN protein immunogen). In some embodiments, a subject was previously treated with one or more additional therapeutic agents prior to being administered one or more therapeutic agents of the present disclosure (e.g. a RAN protein vaccine and/or RAN protein immunogen). In some embodiments, a subject is co-administered one or more additional therapeutic agents at the same time as one or more therapeutic agents of the present disclosure (e.g. a RAN protein vaccine and/or RAN protein immunogen). In some embodiments, a subject is subsequently treated with one or more additional therapeutic agents after being administered one or more therapeutic agents of the present disclosure (e.g. a RAN protein vaccine or RAN protein immunogen). Generally, a therapeutic agent can be a small molecule (e.g., metformin or a metformin derivative), an interfering RNA (e.g., dsRNA, siRNA, miRNA, amiRNA, ASO, aptamer, etc.), protein or fragment thereof, peptide, antibody, such as an anti-RAN protein antibody, etc. In some embodiments, a therapeutic agent modulates RAN protein expression, for example by modulating a pathway that controls RAN protein expression, such as protein kinase R (PKR) pathway, EIF2 pathway, or EIF3 pathway.

The identification and selection of appropriate additional therapeutic agents is within the capabilities of a person of ordinary skill in the art, and will depend upon the disease or disorder from which the subject is suffering. In some embodiments, one or more additional therapeutic agents for the treatment of ALS/FTD (e.g., Riluzole (Rilutek, Sanofi-Aventis), trazodone (Desyrel, Oleptro), selective serotonin reuptake inhibitors (SSRIs), baclofen, diazepam, phenytoin, trihexyphenidyl, amitriptyline, metformin, anti-RAN antibodies, etc.) are administered to the subject. In some embodiments, one or more additional therapeutic agents for HD (e.g., tetrabenazine (Xenazine), baclofen, and deutetrabenazine (Austedo), etc.), AD (e.g., cholinesterase inhibitors (Aricept®, Exelon®, Razadyne®) and memantine (Namenda®), etc.), Fragile X Syndrome (e.g., selective serotonin reuptake inhibitors, carbamazepine, methylphenidate, Trazodone, etc.), Spinocerebellar Ataxia (e.g., baclofen, riluzole, amantadine, varenicline, etc.), Fragile X Syndrome (e.g., sertraline, metformin, cannabidiol (CBD), acamprosate, lovastatin, minocycline, etc.), myotonic dystrophy type 1 (e.g., tideglusib, mexiletine, etc.), or myotonic dystrophy type 2 (e.g., Mexilitene, gabapentin, nonsteroidal anti-inflammatory drugs (NSAIDS), low-dose thyroid replacement, low-dose steroids, tricyclic antidepressants, etc.) are administered to the subject.

A subject may be administered a therapeutically effective amount of one or more therapeutic agents (e.g. a RAN protein vaccine and/or RAN protein immunogen). As used herein, an “effective amount” is a dosage of a therapeutic agent sufficient to provide a medically desirable result, such as treatment or amelioration of one or more signs or symptoms caused by a disease or disorder associated with RAN protein expression, translation, and/or accumulation (e.g., a neurodegenerative disease or disorder). In some embodiments, a therapeutically effective amount is an amount of a vaccine or immunogen sufficient to elicit production of anti-RAN protein antibodies in a subject. In some embodiments, a therapeutically effective amount is an amount effective in increasing the amount of anti-RAN antibodies present in a sample obtained from the subject receiving treatment (e.g., relative to a subject not receiving treatment). In some embodiments, a therapeutically effective amount is an amount effective in reducing repeat expansions in the subject. In some embodiments, a therapeutically effective amount is an amount effective in reducing the transcription of RNAs that produce RAN proteins in a subject. In certain embodiments, a therapeutically effective amount is an amount effective in reducing the translation of RAN proteins in a subject. In certain embodiments, a therapeutically effective amount is an amount effective in reducing RAN protein expression in a subject. In certain embodiments, a therapeutically effective amount is an amount effective in reducing RAN protein aggregation in a subject. “Reducing” expression of a repeat sequence or RAN protein translation refers to a decrease in the amount or level of repeat sequence expression or RAN protein translation in a subject after administration of a therapeutic agent (and relative to the amount or level in the subject prior to the administration).

In certain embodiments, the effective amount is an amount effective in increasing the amount of anti-RAN antibodies present in a sample obtained from the subject receiving treatment by at least 10%, at least 20%, at least 30%, at least 40%, at least 50%, at least 60%, at least 70%, at least 80%, at least 90%, at least 95%, or at least 98% (e.g., the level of anti-RAN antibodies relative to the level of anti-RAN antibodies in a cell or subject that has not been administered a therapeutic agent). In certain embodiments, the effective amount is an amount effective in reducing the level of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative to the level of RAN proteins in a cell or subject that has not been administered a therapeutic agent). In certain embodiments, the effective amount is an amount effective in reducing the translation of RAN proteins by at least 10%, at least 20%, at least 30%, at least 40%, at least 50%, at least 60%, at least 70%, at least 80%, at least 90%, at least 95%, or at least 98% (e.g., the level of RAN proteins relative the level of RAN proteins in a cell or subject that has not been administered a therapeutic agent). In certain embodiments, the effective amount is an amount effective in reducing RAN protein expression by at least 10%, at least 20%, at least 30%, at least 40%, at least 50%, at least 60%, at least 70%, at least 80%, at least 90%, at least 95%, or at least 98% (e.g., the level of RAN proteins relative the level of RAN proteins in a cell or subject that has not been administered a therapeutic agent). In certain embodiments, the effective amount is an amount effective in reducing RAN protein aggregation by at least 10%, at least 20%, at least 30%, at least 40%, at least 50%, at least 60%, at least 70%, at least 80%, at least 90%, at least 95%, or at least 98% (e.g., the level of RAN proteins relative the level of RAN proteins in a cell or subject that has not been administered a therapeutic agent).

The effective amount will vary with the age and physical condition of the subject being treated, the severity of the disease or disorder (e.g., the amount of RAN protein accumulation, or cellular toxicity caused by such an accumulation) in the subject, the duration of the treatment, the nature of any concurrent therapy, the specific route of administration, and other factors within the knowledge and expertise of the health practitioner. An effective amount may be included in a single dose (e.g., single oral dose) or multiple doses (e.g., multiple oral doses).

In some embodiments, a therapeutic agent (e.g., a RAN protein vaccine and/or RAN protein immunogen) is delivered by a viral vector, for example a lentiviral vector, retroviral vector, adenoviral vector, or adeno-associated virus (AAV) vector. In some embodiments, one or more anti-RAN protein peptide vaccines is delivered by one or more rAAVs. In some embodiments, a therapeutic agent is delivered to a subject in a recombinant adeno-associated virus (rAAV) particle.

Administration of a treatment may be accomplished by any method known in the art (see, e.g., Harrison's Principle of Internal Medicine, McGraw Hill, Inc.). Administration may be local or systemic. A therapeutic agent can be administered by any route, including enteral (e.g., oral), parenteral, intravenous, intramuscular, intra-arterial, intramedullary, intrathecal, intratracheal, subcutaneous, intraventricular, transdermal, intradermal, ocular, rectal, intravaginal, intraperitoneal, topical (as by powders, ointments, creams, and/or drops), mucosal, nasal, buccal, sublingual, intratracheal instillation, bronchial instillation, inhalation, as an oral spray, as a nasal spray, and/or as an aerosol. Systemic routes include oral and parenteral. Specifically contemplated routes are oral administration, intravenous administration (e.g., systemic intravenous injection), regional administration via blood and/or lymph supply, and/or direct administration to an affected site. In some embodiments, a treatment as described by the disclosure is administered to a subject by intramuscular injection. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject. Compositions for different routes of administration are well known in the art (see, e.g., Remington's Pharmaceutical Sciences by E. W. Martin).

In some embodiments, a treatment for a disease or disorder associated with RAN protein expression is administered to the central nervous system (CNS) of a subject in need thereof. As used herein, the “central nervous system (CNS)” refers to all cells and tissues of the brain and spinal cord of a subject, including but not limited to neuronal cells, glial cells, astrocytes, cerebrospinal fluid, etc. Modalities of administering a therapeutic agent to the CNS of a subject include direct injection into the brain (e.g., intracerebral injection, intraventricular injection, intraparenchymal injection, etc.), direct injection into the spinal cord of a subject (e.g., intrathecal injection, lumbar injection, etc.), or any combination thereof.

In some embodiments, a treatment as described by the disclosure is systemically administered to a subject, for example by intravenous injection. Systemically administered therapeutic molecules can be modified, in some embodiments, in order to improve delivery of the molecules to the CNS of a subject. Examples of modifications that improve CNS delivery of therapeutic molecules include but are not limited to co-administration or conjugation to blood brain barrier-targeting agents (e.g., transferrin, melanotransferrin, low-density lipoprotein (LDL), angiopeps, RVG peptide, etc., as disclosed by Georgieva et al. Pharmaceuticals 6(4): 557-583 (2014)), coadministration with blood-brain barrier (BBB) disrupting agents (e.g., bradykinins), and physical disruption of the BBB prior to administration (e.g., by MRI-Guided Focused Ultrasound), etc.

Dosage will depend on the subject and the route of administration. Dosage can be determined by the skilled artisan.

A composition (e.g., a composition comprising an isolated nucleic acid, vector, or rAAV) as described by the disclosure can be administered to a subject once or multiple times (e.g., 2, 3, 4, 5, 6, 7, 8, 9, 10, 20, or more than 10) times. In some embodiments, a subject is administered one or more additional doses of a RAN protein vaccine or RAN protein immunogen.

The exact amount of a therapeutic agent required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, eight months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a composition described herein.

Without wishing to be bound by any particular theory, detection (e.g., quantification of anti-RAN protein antibodies) in the biological samples can be used to determine the effectiveness of a therapeutic agent or regime in the subject from which the samples are obtained.

### EXAMPLES

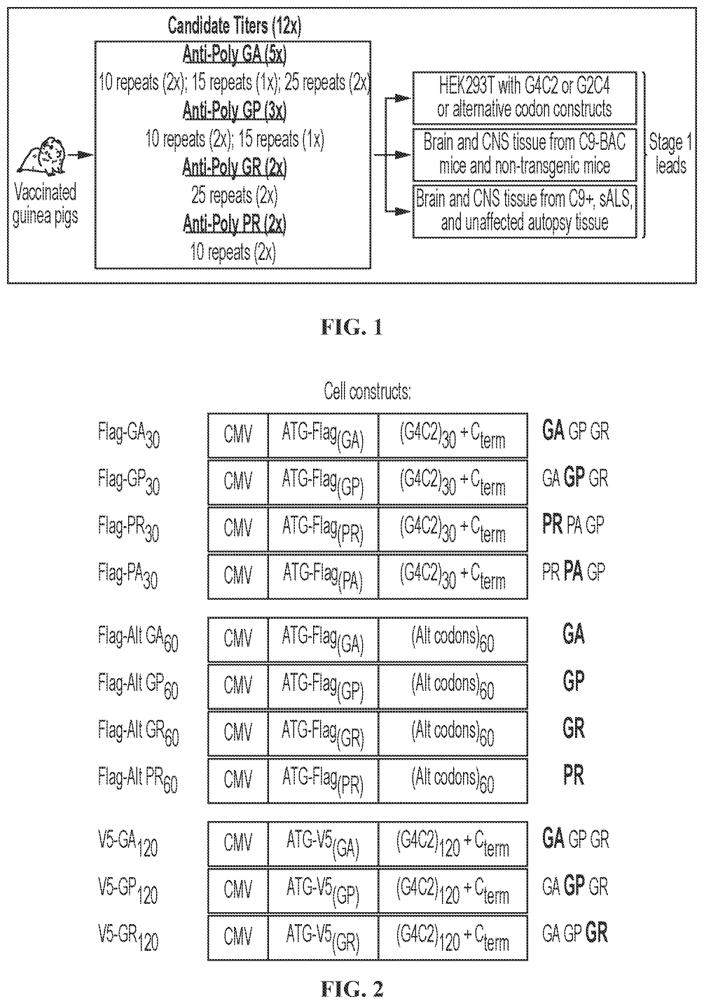

This example describes identification and characterization of peptide-based anti-RAN protein vaccines. FIG. 1 provides a schematic describing a study design.

In Stage 1 of the project, guinea pigs were vaccinated with one of the following anti-RAN protein vaccines, which each target one or more RAN protein di-amino acid repeats: anti-poly(GA), anti-poly(GP), anti-poly(GR), and anti-poly(PR). Some peptide-based vaccines were configured to include one or more heterologous T helper cell (Th) epitopes in order to facilitate production of specific autoantibodies against RAN proteins without eliciting a T-cell response in the subject. Immunogens were linked to the di-amino acid repeat peptides via certain peptide linkers, for example lysine-rich linkers. A summary of vaccines injected into the guinea pigs is provided in Table 1 below.

Animals were intramuscularly injected with 400 μg of peptide/1.0 ml/dose at four injection sites; subsequently, animals were boosted with 100 μg of peptide/0.25 ml/dose intramuscularly. Vaccine and booster doses included peptide and CpG3 (adjuvant) at a 0.7:1 ratio, in 0.2% Tween 80. Sera were collected at 0, 3, 6, 9, 12, and 15 weeks post injection (wpi), and antibody titers were quantified.

TABLE 1
Summary of tested vaccines.

| | | |
|---|---|---|
| Peptide | Peptide repeat contained | SEQ ID |
| Code | in construct | NO: |

GA constructs

| | | |
|---|---|---|
| p4803kb | (GA)₁₀ | 4 |
| p4804kb | (GA)₁₅ | 4 |
| p4805kb | (GA)₁₀ | 4 |
| p4809kb | (GA)₂₅ | 4 |
| p4810kb | (GA)₂₅ | 4 |

GP constructs

| | | |
|---|---|---|
| p4806kb | (GP)₁₀ | 5 |
| p4807kb | (GP)₁₅ | 5 |
| p4808kb | (GP)₁₀ | 5 |

GR constructs

| | | |
|---|---|---|
| p4811kb | (GR)₂₅ | 7 |
| p4812kb | (GR)₂₅ | 7 |

PR constructs

| | | |
|---|---|---|
| p4882kb | (PR)₁₀ | 6 |
| p4883kb | (PR)₁₀ | 6 |

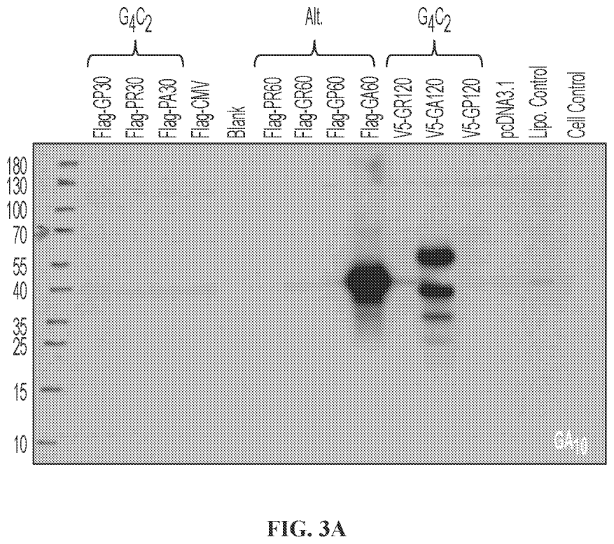

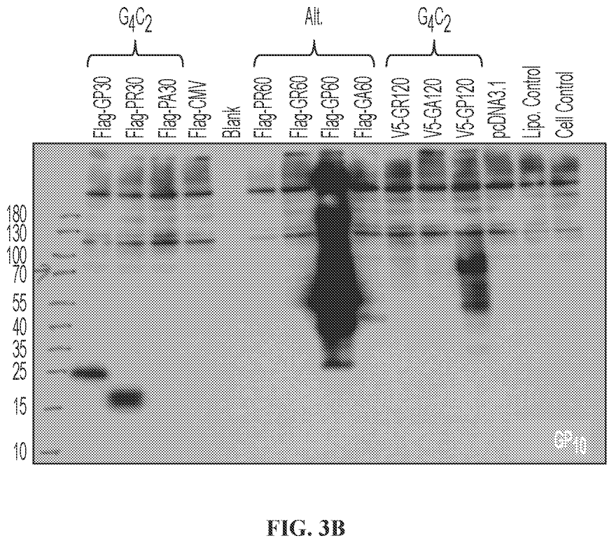

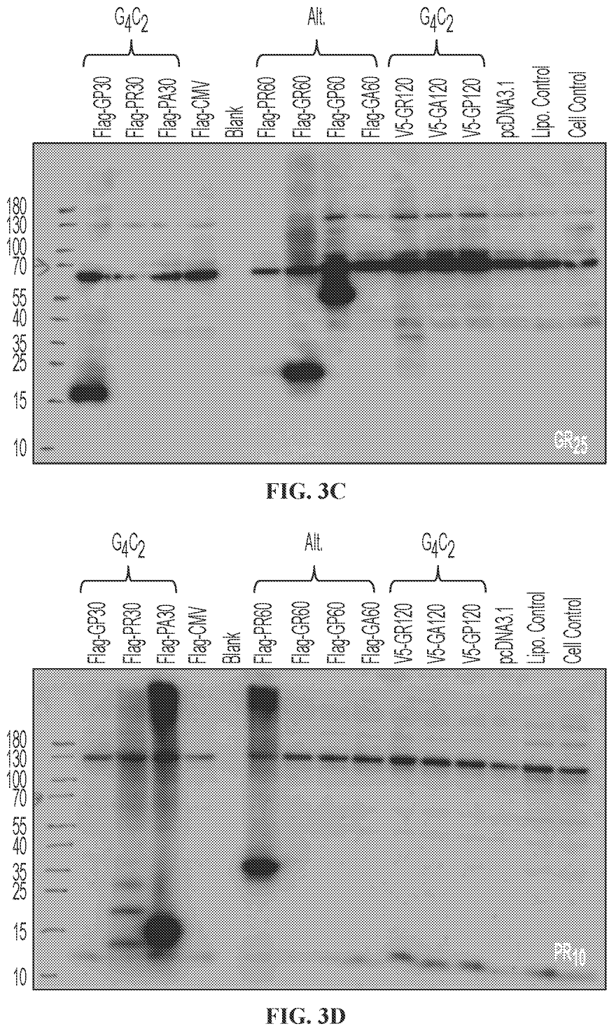

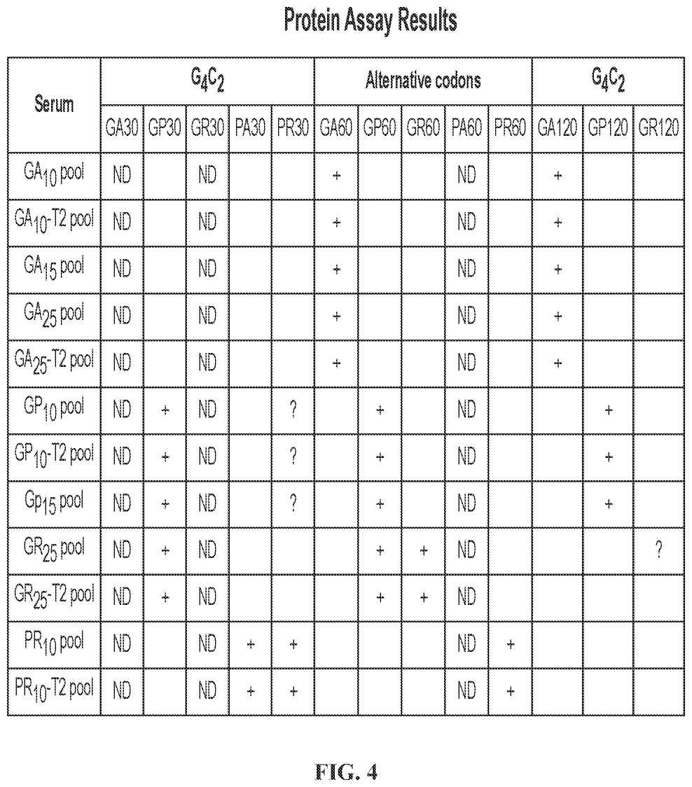

Sera were then tested for the presence and activity of anti-RAN protein antibodies in vitro. Mammalian cells were transfected with expression constructs encoding poly(GA), poly(GR), poly(GP), and poly(PA) RAN proteins having repeat regions of varied lengths (e.g., 30, 60, and 120 repeats in length). Constructs are shown in FIG. 2. RAN proteins were extracted from the cells and run on Western blots that were probed with the vaccinated guinea pig sera. Representative data, shown in FIGS. 3A-3D, indicated that vaccinated guinea pigs produced sera having antibodies that recognize RAN proteins (e.g., di-amino acid repeat containing RAN proteins, such as those translated from C9Orf72 repeat expansions). FIG. 4 provides a summary of protein assay results.

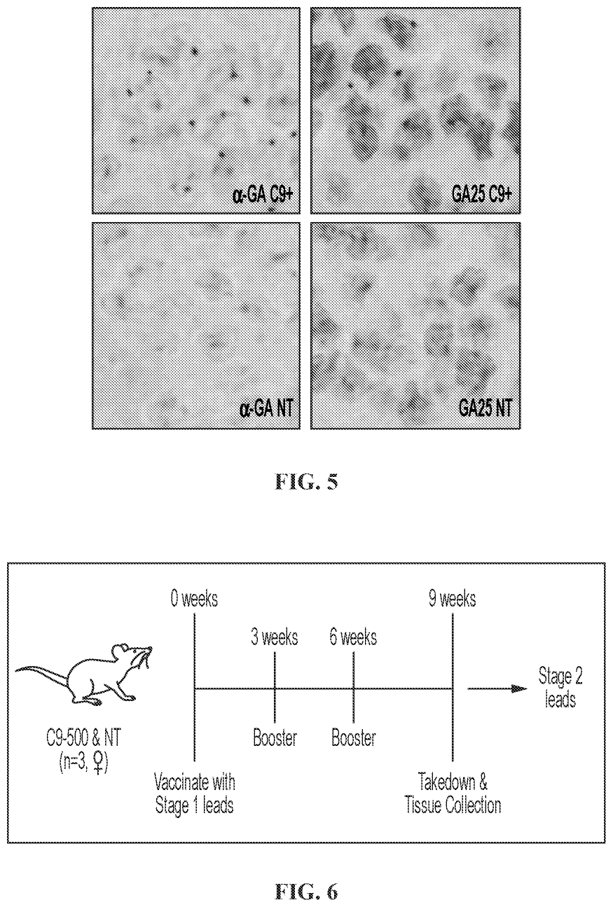

Immunohistochemistry experiments were then performed to assess whether the anti-RAN protein antibodies present in the vaccinated guinea pig sera recognize C9Orf72 di-peptide repeat-containing RAN proteins in situ. Briefly, brain sections from C9+ and control mice were obtained and probed with the vaccinated guinea pig sera. Representative data (FIG. 5) indicate that anti-RAN protein antibodies in the sera (e.g., antibodies produced in GA₂₅ (SEQ ID NO: 4) vaccinated guinea pigs) recognize RAN proteins in situ, staining punctate poly(GA) aggregates in retrosplenial cortex (RSC) of C9+ mice.

In summary, data from Stage 1 indicate that guinea pigs vaccinated with anti-RAN protein vaccines (e.g., anti-poly(GA), anti-poly(GP), anti-poly(GR), anti-poly(PR)) produce high antibody titers of di-peptide repeat (DPR)-recognizing anti-RAN protein antibodies.

In Stage 2, a mouse model of C9Orf72 ALS (termed the “C9-Bac” mouse) was used to investigate vaccination with several anti-RAN protein vaccines. The C9-Bac mouse is described further, for example, by Liu et. al. (2016) Neuron, 90(3):521-34.

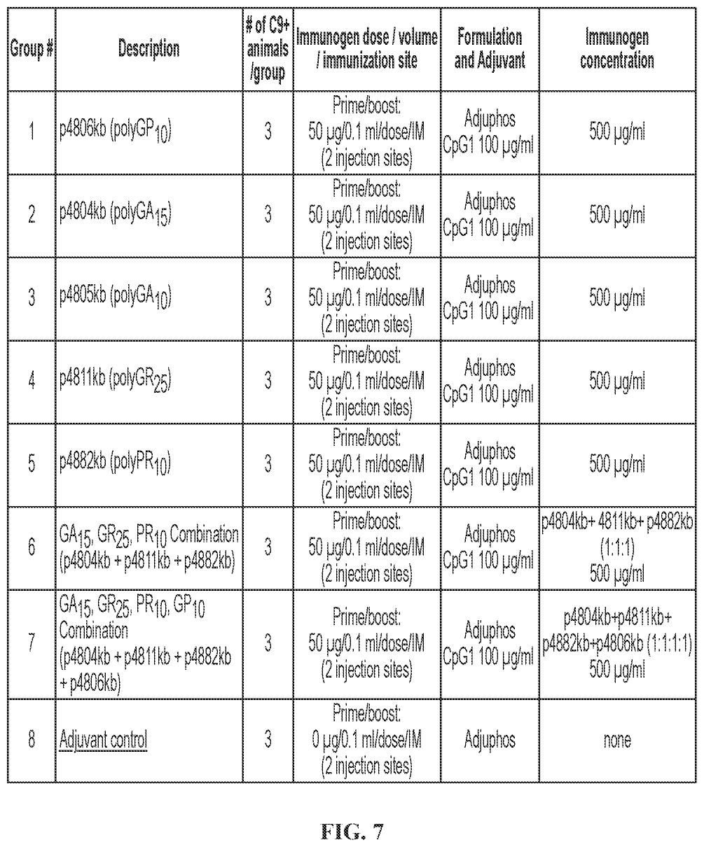

FIG. 6 shows a study design for this example. Briefly, C9-Bac mice were vaccinated with anti-RAN protein vaccines (GA₁₀ (SEQ ID NO: 4), GA₁₅ (SEQ ID NO: 4), GR₂₅ (SEQ ID NO: 7), GP₁₀ (SEQ ID NO: 5), PR₁₀ (SEQ ID NO: 6), and combination vaccines GA₁₅ (SEQ ID NO: 4)+GR₂₅ (SEQ ID NO: 7)+PR₁₀ (SEQ ID NO: 6) and GA₁₅ (SEQ ID NO: 4)+GR₂₅ (SEQ ID NO: 7)+PR₁₀ (SEQ ID NO: 6)+GP₁₀(SEQ ID NO: 5)) at week 0. Booster injections were administered at 3 weeks and 6 weeks post-injection. Animals were taken down at 9 weeks and biological samples were collected. Vaccine and booster doses were administered intramuscularly at an immunogen concentration of 500 μg/ml (50 μg/0.1 ml/dose for prime and boost injections at 2 injection sites). Vaccines were formulated with Adjuphos (aluminum phosphate) and CpG1 adjuvants (100 μg/ml). FIG. 7 provides a summary of vaccine groups in this study.

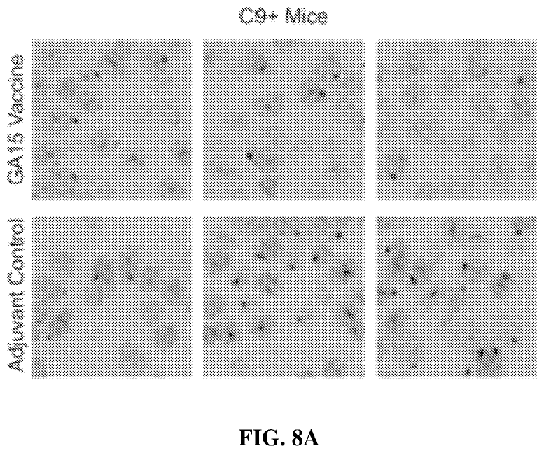

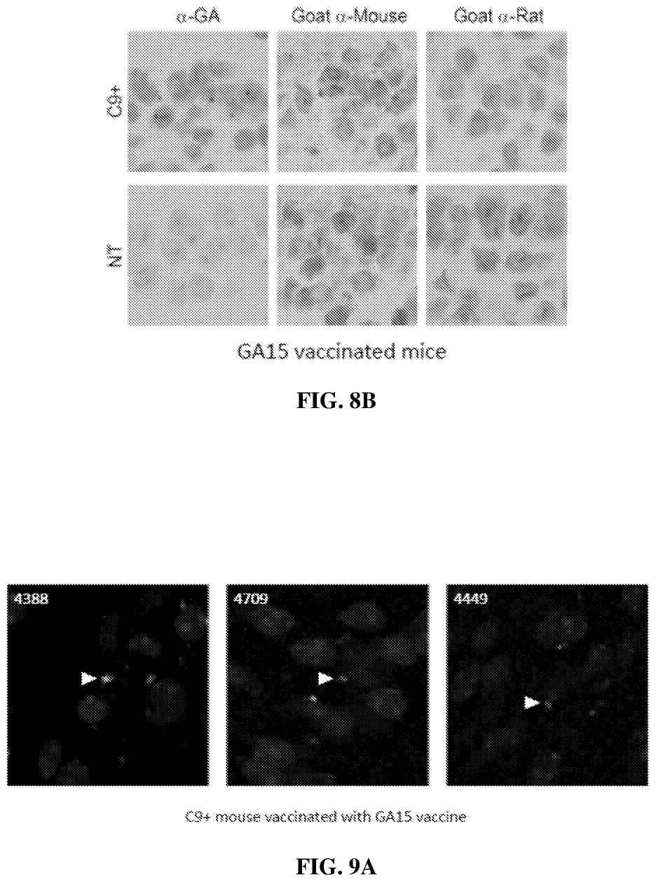

As a control experiment, immunohistochemistry experiments of mouse brains were performed to investigate whether vaccinated mice display similar levels of GA aggregates in the retrosplenial cortex as control animals. Tissue samples were probed with sera from vaccinated mice. FIGS. 8A-8B show representative immunohistochemistry data. FIG. 8A shows poly(GA) RAN protein aggregates in the retrosplenial cortex (RSC) of C9+ mice injected with the GA₁₅ (SEQ ID NO: 4) vaccine or adjuvant injected. FIG. 8B shows mouse anti-RAN protein antibodies are produced in GA₁₅ (SEQ ID NO: 4) vaccinated mice and form puncta in the RSC of C9+ mice, but not NT mice.

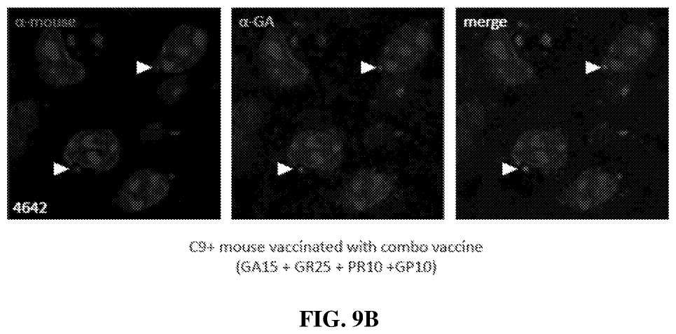

Recognition of di-peptide repeat (DPR) RAN proteins in C9-Bac mice by antibodies produced in vaccinated mice was also investigated. Representative immunofluorescence data (FIGS. 9A-9B) show representative fluorescence microscopy data indicating anti-RAN protein antibodies produced by vaccinated mice colocalized with GA aggregates detected in the brain with anti-rabbit antibodies. FIG. 9A shows data for a C9+ mouse vaccinated with GA₁₅ (SEQ ID NO: 4) vaccine. FIG. 9B shows data for a C9+ mouse vaccinated with a combination (GA₁₅ (SEQ ID NO: 4)+GR₂₅ (SEQ ID NO: 7)+PR₁₀ (SEQ ID NO: 6)+GP₁₀ (SEQ ID NO: 5)) vaccine, indicating that anti-RAN protein antibodies recognize di-peptide repeat RAN proteins in situ.

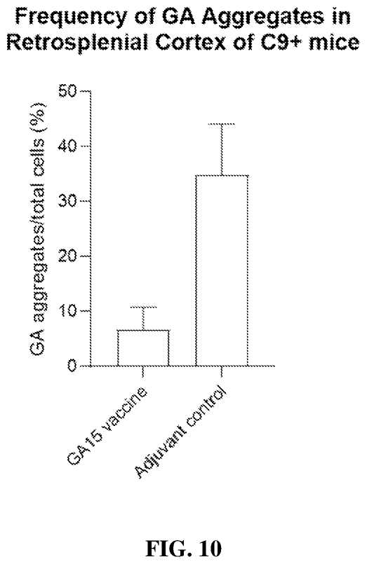

FIG. 10 shows that GA aggregates were reduced in mice treated with the GA₁₅ (SEQ ID NO: 4) vaccine. 6 μm brain sections from C9(+) mice treated with either the GA₁s (SEQ ID NO: 4) vaccine (n=4) or adjuvant control (n=3) were stained by immunohistochemistry for the presence of GA aggregates using an anti-GA antibody and a hematoxylin counterstain. Total GA aggregates in the retrosplenial cortex of C9(+) mice treated with GA₁s (SEQ ID NO: 4) vaccine (n=4) or adjuvant control (n=3). Data are presented as GA aggregates/total cells (n=3 animals/group).

1. A method for preventing or treating a disease or disorder associated with RAN proteins in a human subject in need thereof, the method comprising administering to the subject a therapeutically effective amount of a RAN protein vaccine, wherein administration of the RAN protein vaccine to the subject elicits production of one or more anti-RAN protein antibodies in the subject.

2. The method of claim 1, wherein the disease or disorder associated with RAN proteins is amyotrophic lateral sclerosis (ALS), Huntington's disease (HD), Fragile X Syndrome (FRAXA), Spinal Bulbar Muscular Atrophy (SBMA), Dentatorubropallidoluysian Atrophy (DRPLA), Spinocerebellar Ataxia 1 (SCA1), Spinocerebellar Ataxia 2 (SCA2), Spinocerebellar Ataxia 3 (SCA3), Spinocerebellar Ataxia 6 (SCA6), Spinocerebellar Ataxia 7 (SCA7), Spinocerebellar Ataxia 8 (SCA8), Spinocerebellar Ataxia 12 (SCA12), or Spinocerebellar Ataxia 17 (SCA17), amyotrophic lateral sclerosis (ALS), Spinocerebellar ataxia type 36 (SCA36), Spinocerebellar ataxia type 29 (SCA29), Spinocerebellar ataxia type 10 (SCA10), myotonic dystrophy type 1 (DM1), myotonic dystrophy type 2 (DM2), or Fuch's Corneal Dystrophy (CTG181).

3. The method of claim 2, wherein the disease is:
(a) ALS, and wherein the ALS is caused by one or more mutations in a C9Orf72 gene;
(b) HD, and wherein the HD is caused by one or more mutations in a Htt gene;
(c) AD, and wherein the AD is caused by one or more mutations in an LRP8, CASP8, and/or GREB1 gene;
(d) SCA36, and wherein the SCA36 is caused by one or more mutations in an SCA36 gene; or
(e) FTD, and wherein the FTD is caused by one or more mutations in a C9Orf72 gene.

4. The method of claim 1, wherein cells of the subject translate one or more RAN proteins.

5. The method of claim 4, wherein the RAN proteins being translated in the subject:
(a) comprise one or more of the following: poly(CP), poly(GA), poly(GP), poly(PR), poly(GR), poly(PA), poly(A), poly(G), poly(S), poly(C), poly(Q), poly(GD), poly(GE), poly(GQ), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), or poly(GK); and/or
(b) are poly(GA), poly(GP), poly(GR), poly(PA), and/or poly(PR) RAN proteins expressed from a C9Orf72 expansion repeat of the subject.

6. (canceled)

7. The method of claim 1, wherein the RAN protein vaccine comprises one or more di-peptide repeat (DPR) peptide antigens.

8. The method of claim 7, wherein each DPR peptide antigen comprises between 2 di-amino acid repeats and 150 di-amino acid repeats, optionally between:
(a) 10 di-amino acid repeats and 25 di-amino acid repeats;
(b) 25 di-amino acid repeats and 50 di-amino acid repeats;
(c) 50 di-amino acid repeats and 100 di-amino acid repeats; or
(d) 100 di-amino acid repeats and 150 di-amino acid repeats.

9. (canceled)

10. The method of claim 7, wherein:
(a) each of the one or more DPR peptide antigens comprises a (GA)ₓ (SEQ ID NO: 4), (GP)ₓ (SEQ ID NO: 5), (GR)ₓ (SEQ ID NO: 7), (PA)ₓ (SEQ ID NO: 8), (PR)ₓ (SEQ ID NO: 6), (CP)ₓ (SEQ ID NO:3), (A)ₓ (SEQ ID NO:9), (G)ₓ (SEQ ID NO:10), (S)ₓ (SEQ ID NO: 11), (C)ₓ (SEQ ID NO: 12), (Q)ₓ (SEQ ID NO: 13), (GD)ₓ (SEQ ID NO: 14), (GE)ₓ (SEQ ID NO: 15), (GQ)ₓ (SEQ ID NO:16), (GT)ₓ (SEQ ID NO:17), (L)ₓ (SEQ ID NO: 18), (LP)ₓ (SEQ ID NO: 19), (LPAC)_{X} (SEQ ID NO: 20), (LS)ₓ (SEQ ID NO: 21), (P)ₓ (SEQ ID NO: 22), (QAGR)_{X} (SEQ ID NO: 23), (RE)ₓ (SEQ ID NO: 24), (SP)ₓ (SEQ ID NO: 25), (VP)ₓ (SEQ ID NO: 26), (FP)ₓ (SEQ ID NO: 27), and/or (GK)ₓ (SEQ ID NO: 28) di-amino acid repeat, wherein x represents the number of repeat units of the antigen, and wherein x is 5, 10, 15, 20, 25, 30, 35, or 40;
(b) the DPR peptide antigen comprises (GA)₁₀(SEQ ID NO: 4), (GA)₁₅ (SEQ ID NO: 4), (GA)₂₅ (SEQ ID NO: 4), (GR)₂₅ (SEQ ID NO: 7), (GP)₁₀ (SEQ ID NO: 5), (GP)₁₅ (SEQ ID NO: 5), (PR)₁₀(SEQ ID NO: 6), or a combination thereof; or
(c) the DPR peptide antigen comprises [(GA)₁₅ (SEQ ID NO: 4)+(GR)₂₅ (SEQ ID NO: 7)+(PR)₁₀(SEQ ID NO: 6)] or [(GA)₁₅ (SEQ ID NO: 4)+(GR)₂₅ (SEQ ID NO: 7)+(PR)₁₀(SEQ ID NO: 6)+(GP)₁₀ (SEQ ID NO: 5)].

11-12. (canceled)

13. The method of claim 7, wherein the RAN protein vaccine further comprises one or more additional immunogens.

14. The method of claim 13, wherein the one or more immunogens:
(a) comprises keyhole limpet hemocyanin (KLH), Blue Carrier Immunogenic protein (CCH), bovine serum albumin (BSA), ovalbumin (OVA), diphtheria toxin, measles virus fusion protein (MVF), hepatitis B virus surface antigen (HB-sAg), tetanus toxin (TT), pertussis toxin (PT), a T-cell helper epitope, or a portion of any of the foregoing; and/or
(b) are linked to the DPR peptide antigen via one or more linking molecules.

15. (canceled)

16. The method of claim 14, wherein each of the one or more the linking molecules is selected from the group consisting of: an amino acid, Lys-, Gly-, and Lys-Lys-Lys.

17-18. (canceled)

19. The method of claim 1, wherein:
(a) each of the one or more anti-RAN protein antibodies bind to a poly(CP), poly(GA), poly(GP), poly(PR), poly(GR), poly(PA), poly(A), poly(G), poly(S), poly(C), poly(Q), poly(GD), poly(GE), poly(GO), poly(GT), poly(L), poly(LP), poly(LPAC (SEQ ID NO: 1)), poly(LS), poly(P), poly(QAGR (SEQ ID NO: 2)), poly(RE), poly(SP), poly(VP), poly(FP), or poly(GK) di-amino acid repeat-containing RAN protein; and/or
(b) the anti-RAN protein antibodies are one or more of an anti-poly(CP), anti-poly(GA), anti-poly(GP), anti-poly(PR), anti-poly(GR), anti-poly(PA), anti-poly(A), anti-poly(G), anti-poly(S); anti-poly(C); anti-poly(Q); anti-poly(GD), anti-poly(GE), anti-poly(GQ), anti-poly(GT), anti-poly(L), anti-poly(LP), anti-poly(LPAC (SEQ ID NO: 1)), anti-poly(LS), anti-poly(P), anti-poly(QAGR (SEQ ID NO: 2)), anti-poly(RE), anti-poly(SP), anti-poly(VP), anti-poly(FP), and/or anti-poly(GK) antibody.

20. The method of claim 1, wherein the subject is administered one or more additional doses of the RAN protein vaccine.

21-28. (canceled)

29. The method of claim 1, wherein the RAN protein vaccine comprises a B-cell epitope.

30. The method of claim 1, wherein administration of the RAN protein vaccine reduces:
(a) RAN protein expression in the subject, relative to the level of RAN protein expression present in the subject prior to administration; and/or
(b) RAN protein aggregation in the subject, relative to the level of RAN protein aggregation present in the subject prior to administration.

31. The method of claim 30, wherein RAN protein expression and/or aggregation is reduced by 1%, 5%, 10%, 15%, 20%, 25%, 30%, 35%, 40%, 45%, 50%, 55%, 60%, 65%, 70%, 75%, 80%, 85%, 90%, 95%, or 100% after administration of the RAN protein vaccine to the subject, relative to the level of RAN protein expression and/or aggregation present in the subject prior to administration.

32-33. (canceled)

34. The method of claim 1, wherein the RAN protein vaccine further comprises one or more adjuvants.

35. The method of claim 34, wherein the one or more adjuvants comprise any one or more of: immune targeting adjuvants; immunomodulating adjuvants; incomplete Freund's adjuvant; aluminum phosphate; aluminum hydroxide; alum; Stimulon® QS-21; MPL®; interleukin-12; an oil formulation; a polymer; a micelle forming adjuvant; a saponin; an immunostimulating complex matrix (ISCOM matrix); a particle; DDA; DNA adjuvants; an encapsulating adjuvant; flagellin adjuvants; alhydrogel (Al(OH)₃); CpG1; CpG3 and/or adjuphos (AlPO₄).

36. The method of claim 1, wherein the RAN protein vaccine is delivered to the subject in a composition comprising an exosome or in a composition comprising a nanoparticle, optionally a lipid nanoparticle.

37-38. (canceled)

39. The method of claim 1, wherein the RAN protein vaccine is an mRNA vaccine.
